SEQUENCE LISTING (47 sequences)

SEQ ID NO: 1 - DNA, 20 bp, Artificial Sequence
  Synthetic Construct
ccggtagcacaccttgtaat

SEQ ID NO: 2 - DNA, 20 bp, Artificial Sequence
  Synthetic Construct
agttgctggtgcatgtagaa

SEQ ID NO: 3 - DNA, 24 bp, Artificial Sequence
  Synthetic Construct
  misc_feature (17)..(17): Locked Nucleic Acid
tggtttccaacccacttatggtgt

SEQ ID NO: 4 - DNA, 16 bp, Artificial Sequence
  Synthetic Construct
tggtttccaacccact

SEQ ID NO: 5 - DNA, 21 bp, Artificial Sequence
  Synthetic Construct
cgtggtgtttattaccctgac

SEQ ID NO: 6 - DNA, 24 bp, Artificial Sequence
  Synthetic Construct
caccatcattaaatggtaggacag

SEQ ID NO: 7 - DNA, 28 bp, Artificial Sequence
  Synthetic Construct
tacttggttccatgctatctctgggacc

SEQ ID NO: 8 - DNA, 31 bp, Artificial Sequence
  Synthetic Construct
cctgtatagattgtttaggaagtctaatctc

SEQ ID NO: 9 - DNA, 21 bp, Artificial Sequence
  Synthetic Construct
agttgctggtgcatgtagaag

SEQ ID NO: 10 - DNA, 29 bp, Artificial Sequence
  Synthetic Construct
agcacaccttgtaatggtgttaaaggttt

SEQ ID NO: 11 - DNA, 26 bp, Artificial Sequence
  Synthetic Construct
agcacaccttgtaatggtgttaaagg

SEQ ID NO: 12 - DNA, 23 bp, Artificial Sequence
  Synthetic Construct
aggtgatgaagtcagacaaatcg

SEQ ID NO: 13 - DNA, 19 bp, Artificial Sequence
  Synthetic Construct
caagctataacgcagcctg

SEQ ID NO: 14 - DNA, 26 bp, Artificial Sequence
  Synthetic Construct
cagggcaaactggaacgattgctgat

SEQ ID NO: 15 - DNA, 26 bp, Artificial Sequence
  Synthetic Construct
cagggcaaactggaaacattgctgat

SEQ ID NO: 16 - DNA, 18 bp, Artificial Sequence
  Synthetic Construct
tgaagacctcacagtaaa

SEQ ID NO: 17 - DNA, 27 bp, Artificial Sequence
  Synthetic Construct
tctagctacagagaaatctcgatggag

SEQ ID NO: 18 - DNA, 18 bp, Artificial Sequence
  Synthetic Construct
ctgtccagtcatcaattc

SEQ ID NO: 19 - DNA, 22 bp, Artificial Sequence
  Synthetic Construct
taaggcctgctgaaaatgactg

SEQ ID NO: 20 - DNA, 18 bp, Artificial Sequence
  Synthetic Construct
cacgtctgcagtcaactg

SEQ ID NO: 21 - DNA, 22 bp, Artificial Sequence
  Synthetic Construct
cctacgccacaagctccaacta

SEQ ID NO: 22 - DNA, 21 bp, Artificial Sequence
  Synthetic Construct
ggtcctgcaccagtaatatgc

SEQ ID NO: 23 - DNA, 20 bp, Artificial Sequence
  Synthetic Construct
gctgtatcgtcaaggcactc

SEQ ID NO: 24 - DNA, 17 bp, Artificial Sequence
  Synthetic Construct
caaagggcatgaactac

SEQ ID NO: 25 - DNA, 18 bp, Artificial Sequence
  Synthetic Construct
tttgggcgggccaaactg

SEQ ID NO: 26 - DNA, 17 bp, Artificial Sequence
  Synthetic Construct
cactctggtgggtatag

SEQ ID NO: 27 - DNA, 17 bp, Artificial Sequence
  Synthetic Construct
ccctcaacaagatgttt

SEQ ID NO: 28 - DNA, 24 bp, Artificial Sequence
  Synthetic Construct
tggtttccaacccacttatggtgt

SEQ ID NO: 29 - DNA, 22 bp, Artificial Sequence
  Synthetic Construct
taaggcctgctgaaaatgactg

SEQ ID NO: 30 - DNA, 19 bp, Artificial Sequence
  Synthetic Construct
aggttgtgaggcactgccc

SEQ ID NO: 31 - DNA, 17 bp, Artificial Sequence
  Synthetic Construct
cttccactcggataaga

SEQ ID NO: 32 - DNA, 24 bp, Artificial Sequence
  Synthetic Construct
cagggcaaactggaacgattgctg

SEQ ID NO: 33 - DNA, 29 bp, Artificial Sequence
  Synthetic Construct
agcacaccttgtaatggtgttaaaggttt

SEQ ID NO: 34 - DNA, 26 bp, Artificial Sequence
  Synthetic Construct
tgtaattagaggtgatgaagtcagac

SEQ ID NO: 35 - DNA, 19 bp, Artificial Sequence
  Synthetic Construct
caagctataacgcagcctg

SEQ ID NO: 36 - DNA, 19 bp, Artificial Sequence
  Synthetic Construct
ttgctggtgcatgtagaag

SEQ ID NO: 37 - DNA, 21 bp, Artificial Sequence
  Synthetic Construct
  misc_feature (10)..(10): Locked Nucleic Acid
ctacgccaccagctccaacta

SEQ ID NO: 38 - DNA, 21 bp, Artificial Sequence
  Synthetic Construct
ctacgccaccagctccaacta

SEQ ID NO: 39 - DNA, 20 bp, Artificial Sequence
  Synthetic Construct
caggctgcgttatagcttgg

SEQ ID NO: 40 - DNA, 33 bp, Artificial Sequence
  Synthetic Construct
tgagattagacttcctaaacaatctataccggt

SEQ ID NO: 41 - DNA, 21 bp, Artificial Sequence
  Synthetic Construct
tcaggccggtagcaaaccttg

SEQ ID NO: 42 - DNA, 20 bp, Artificial Sequence
  Synthetic Construct
tgtgacatacccattggtgc

SEQ ID NO: 43 - DNA, 23 bp, Artificial Sequence
  Synthetic Construct
gattgactagctacactacgtgc

SEQ ID NO: 44 - DNA, 26 bp, Artificial Sequence
  Synthetic Construct
atcagactcagactaagtctcatcgg

SEQ ID NO: 45 - DNA, 23 bp, Artificial Sequence
  Synthetic Construct
caagactcactttcttccacagc

SEQ ID NO: 46 - DNA, 22 bp, Artificial Sequence
  Synthetic Construct
cctcaactttgtcaagacgtga

SEQ ID NO: 47 - DNA, 22 bp, Artificial Sequence
  Synthetic Construct
  misc_feature (14)..(14): Locked Nucleic Acid
atgtggtcaaccataatgcaca

(19) United States
(12) Patent Application Publication
(10) Pub. No.: US 2022/0389488 A1
(43) Pub. Date: Dec. 8, 2022

(54) MULTIPLEXED GENOTYPING ASSAYS WITH A SINGLE PROBE USING FLUORESCENT AMPLITUDE TUNING

(71) Applicant: GT MOLECULAR, LLC, Fort Collins, CO (US)

(72) Inventors: Sarah KANE, Fort Collins, CO (US); Rose NASH, Fort Collins, CO (US); Christopher McKEE, Fort Collins, CO (US)

(21) Appl. No.: 17/693,129

(22) Filed: Mar. 11, 2022

Related U.S. Application Data
(60) Provisional application No. 63/160,432, filed on Mar. 12, 2021.
(60) Provisional application No. 63/172,839, filed on Apr. 9, 2021.

(51) Int. Cl. C12Q 1/6827; C12Q 1/686; C12Q 1/6853

(52) U.S. Cl. CPC C12Q 1/6827; C12Q 1/686; C12Q 1/6853

(57) ABSTRACT

Provided are methods and kits for detecting and quantifying a sequence difference relative to a target polynucleotide sequence, and methods for developing assays. Target polynucleotide sequences are examined to determine if any of the sequences vary by at least one nucleotide difference. Promiscuous probes that have on and off-target binding at different binding efficiencies at permissive temperature are used, so that differences in polynucleotide sequences are reliably detected and quantified in a single well with fewer types of labeled probes, including by polymerase chain reaction of any of a range of sequences where there is interest in detecting an at least one nucleotide difference relative. The target sequences may be a reference polynucleotide sequence indicative of a first state, such as “normal” and another sequence that varies by at least one polynucleotide indicative of a different second state, such as a mutation, disease condition, or predisposition thereto.

### CROSS-REFERENCE TO RELATED APPLICATIONS

This application claims benefit of U.S. Pat. App. Nos. 63/160,432 filed Mar. 12, 2021 and 63/172,839 filed Apr. 9, 2021, each of which are hereby incorporated by reference to the extent not inconsistent herewith.

### REFERENCE TO A SEQUENCE LISTING

A sequence listing containing SEQ ID NOs: 1-47 in computer readable form is submitted herewith and is specifically incorporated by reference. That sequence listing is supported by the specification and does not go beyond the disclosure in this Application as filed.

### BACKGROUND OF INVENTION

There continues to be a need for the collection of sampled polynucleotide sequence information which is highly informative in various contexts including clinical and diagnostic contexts, environmental testing, wastewater-based epidemiology, agribiotechnology contexts, and numerous other economically important areas. Sample testing of for polynucleotide sequence information can be resource intensive because a variety of reagents are required for performance of molecular assays on test samples. For example, in the context of PCR assays, the needed reagents commonly include amplification primers, detectable probes, polymerase enzyme, PCR buffering reagents, etc. Further, vessels are needed for sample testing such as tubes or wells of plates.

In view of the resource intensive nature of polynucleotide sequence information collection, there continues to be a need in the art for methods which utilize testing supplies efficiently, whether the supplies are reagents, such as PCR reagents, probes, or vessels in which to run reactions. Provided herein are methods and kits for molecular assays designed to collect polynucleotide sequence information which provide for efficiencies in the supplies needed for testing, particularly with respect to a decrease in number of fluorophores, while providing an ability to distinguish between sequences having as little as one nucleotide difference. The methods and kits provided herein reduce the number of fluorophores required to detect various target polynucleotide sequences, by fluorescent amplitude tuning associated with on- and off-target binding of a promiscuous probe to a target polynucleotide sequence with and without a change in the sequence, such as due to a mutation. The amplitude tuning is facilitated by different binding efficiencies between the promiscuous probe within a permissive temperature range to the target polynucleotide sequences, with lower binding efficiency associated with a lower fluorescent amplitude output relative to the higher binding efficiency having a relatively higher fluorescent amplitude output.

In addition, genotyping can be extremely challenging, particularly for applications where there is only very minor changes between nucleic acids. In such situations, non-specific amplification can present challenges to robust and sensitive genotyping. Furthermore, conventional genotyping assays are rather reagent intensive, with two or more wells used with two or more fluorophores/probes per well in order to test for a wildtype (WT) a housekeeping gene, and sequences containing one or more mutations that make the sequences different from the WT. That configuration, therefore, requires the use of more probes and reagents and wells in a plate for each sample. Provided herein are methods, assays and/or kits that address such problems by accommodating off-target amplification, albeit at less efficiency, thereby discriminating nucleotide mismatches to reduce the need for additional reagents and the total number of wells.

### SUMMARY OF THE INVENTION

The present disclosure provides methods and kits for the targeted collection of polynucleotide sequence information. In certain aspects, methods and kits are disclosed wherein labeled probes provide for the detection of the presence or absence of at least one nucleotide difference between a first polynucleotide sequence and a second polynucleotide sequence. In certain aspects, methods and kits disclosed herein provide for multiplexing of molecular assays which result in efficiencies in the utilization of testing supplies, such as PCR reagents, as well as containers in which PCR reactions are run, including wells in a multi-well plate.

The methods and kits provided herein are useful for detecting and quantifying a sequence difference relative to a reference polynucleotide sequence, including as small as one nucleotide difference. Generally, target polynucleotide sequences are examined to determine if any of the sequences vary by an at least one nucleotide difference. For a sequence that does have a relevant change with a use to identify and quantify in a test, probes and primers are accordingly designed. The methods provided herein rely on such probes and primers to, as desired, detect and/or quantify these differences in nucleotide sequences, including identifying whether one or both of the sequences are present in a sample. This can be of particular use when the target polynucleotide sequence is from a virus, because viruses can have relatively high rate of mutation. In such a situation, the at least one nucleotide difference may correspond to a variant, and it is desirable to quantify the amount of variant relative to a parental or wild-type target polynucleotide sequence. Of course, the methods provided herein are compatible with any type of sequence from any source or sample, so long as there an at least one nucleotide difference between the polynucleotides.

One functional benefit of the methods and kits provided herein is the ability to identify and quantify different target polynucleotide sequences, such as a first sequence corresponding to a “parental” (e.g., “normal” or “wild-type”) sequence and a second sequence having at least one nucleotide difference from the first sequence. The methods and kits are efficient, reliable and cost-effective platforms for identifying a change, such as a mutation, in a polynucleotide sequence, without having to use an additional fluorophore. As described herein, this is achieved by specific selection of primers and a corresponding promiscuous probe that can bind both amplicons, with a first amplicon from PCR amplification of a first target polynucleotide sequence (e.g., the parental sequence) and a second target polynucleotide sequence associated having at least one nucleotide difference. The primers are sufficiently far from the at least one nucleotide difference so that amplicons associated with either target polynucleotide sequence are produced via PCR without having to vary the sequence of the primer pair. The promiscuous probe at a permissive temperature while able to bind to either amplicon, however, has different binding efficiencies to each of the two amplicons (one having the parental sequence between the primers and the other having the parental sequence between the primers but with the at least one nucleotide difference). The methods and kits exploit this difference in binding efficiency to be able to optically distinguish between the first and second polynucleotide sequences from the single promiscuous probe, such as by detection of fluorescent amplitude by instruments having optical detectors. The promiscuous probe having the higher binding efficiency to one amplicon results in a higher optical signal the that same promiscuous probe with a correspondingly lower binding efficiency to the second amplicon. Further multiplexing is available by introducing one or more additional promiscuous probes relevant for a different nucleotide difference, such as a different mutation at a different loci. For example, a fluorescent label having a different emission spectrum can be readily optically identified by use of appropriate filters with an optical detector. In cases where a probe hybridization region shows differing mutations between samples, the amplitude tuning can further be used to identify the type of mismatch (i.e. G to A vs G to C, etc.).

In an embodiment, provided herein are methods for detecting the presence or absence of an at least one nucleotide difference in a target polynucleotide sequence. The method may comprise the steps of: providing a set of PCR primers, with at least two primers that provide a forward amplification primer and a reverse amplification primer which, when hybridized to the respective primer annealing sites, flank the location of the target polynucleotide sequence. The set of PCR primers are configured to generate in a PCR reaction a first amplicon comprising at least a portion of the target polynucleotide sequence and a second amplicon comprising at least a portion of the target polynucleotide with the at least one nucleotide difference. In other words, one single primer pair comprising a forward and revers primer can be used to amplify two amplicons (one amplicon corresponding to the “original” sequence and the second amplicon to a sequence having an at least one nucleotide difference from the original sequence). A promiscuous probe is provided that at a permissive temperature hybridizes at a first hybridization efficiency to the first amplicon and a second hybridization efficiency to the second amplicon, wherein the first hybridization efficiency is different than the second hybridization efficiency. In this manner, it does not matter whether the higher efficiency is to the original nucleotide or to the nucleotide having the at least one nucleotide difference, so long the hybridization efficiencies are sufficiently different that there will be an optically-distinguishable output. A sample PCR reaction mixture is provided comprising: a test sample having a sample polynucleotide, the PCR primers, the promiscuous probe, and PCR reagents. At least one PCR reaction is performed on the sample PCR reaction mixture at the permissive temperature to generate sample amplicons having promiscuous probe bound thereto. Fluorescence output generated by the promiscuous probe bound to the sample amplicons is measured at the permissive temperature, wherein the probe can bind to both amplicons, wherein the difference between the first and second hybridization efficiencies results in a promiscuous probe fluorescence amplitude difference between promiscuous probe bound to target polynucleotide sequences with and without the at least one nucleotide difference. In this manner, the presence or absence of the at least one nucleotide difference in the target polynucleotide sequence is detected. This method is particularly useful for samples that may have the target polynucleotide sequence, but may or may not also have the other target polynucleotide sequence with the at least one nucleotide difference.

The measuring step may further comprise quantifying the amount of the target polynucleotide sequence having the at least one nucleotide difference. in other words, the methods and kits may be used to quantify a target polynucleotide sequence.

The method may further comprise the steps of using one or more positive controls, including to verify the method is working properly. For example, the method may further comprise: preparing a positive control reaction mixture comprising the following constituents: a positive control mixture comprising a first polynucleotide having the target polynucleotide sequence without the at least one nucleotide difference; a positive control mixture comprising a second polynucleotide having the target polynucleotide sequence and the at least one nucleotide difference; and a positive control mixture comprising the first polynucleotide and the second polynucleotide (for aspects where one probe identifies three targets, a mixture of all three targets can be provided). The method may then involve contacting each of the positive control reaction mixture constituents individually with the set of PCR primers and the promiscuous probe; performing at least one PCR reaction on each of the contacted control reaction mixture constituents to generate a first and/or a second positive amplicon for each of the three constituent positive control reaction mixtures; and validating the method by measuring a positive control fluorescence output generated by the promiscuous probe bound to the first and/or second positive amplicons, wherein a positive validation corresponds to positive clustering of fluorescence output. Of course, for higher multiplexing, additional target polynucleotide sequences are provided. For one promiscuous probe used to identify three targets, there is an additional polynucleotide, a third polynucleotide. Accordingly, a positive control mixture may comprise all the polynucleotides, such as the first, second and third polynucleotides, and so on.

The method may further comprise the step of: defining a target threshold from the positive control mixture. The target threshold may correspond to a clustering signature, defining upper and lower limits from multiple channels.

The method may further comprise the validating that provides: (i) validation of each component of the method; and (ii) threshold definitions for each of the first polynucleotide and the second polynucleotide.

The positive control mixture may comprise between 50-500 copies/μL of the first polynucleotide and/or between 50-500 copies/μL of the second polynucleotide.

The methods are compatible with any of a range of target polynucleotide sequences, including from a range of organisms. For example, the target polynucleotide sequence may be from an organism selected from the group consisting of a virus, a bacteria, a fungus, a parasite, a plant cell, and an animal cell. The cell may be a cancer cell.

The target polynucleotide may comprise RNA, and the method may further comprise the step of performing a reverse transcription reaction to produce a DNA target polynucleotide.

The at least one nucleotide difference may be from a mutation of one or more nucleotides in the target polynucleotide sequence, including an insertion mutation, a deletion mutation, and a single nucleotide polymorphism (SNP).

The method and kits are compatible with any of a range of target polynucleotide sequence lengths. In an aspect, the length selected from a range of 60 bps to 1500 bps.

The test sample may comprise an environmental sample, soil, seed, plant material, wastewater sample, industrial water sample, natural water sample (including river, lake, stream, ocean, groundwater, well water, aquifer), a biological sample such as a gut/stool sample, a liquid or tumor biopsy from a cancer patient, a swab or saliva sample, or an animal sample (veterinary/animal husbandry).

The test sample may be analyzed for short or single nucleotide polymorphisms (SNPs) whether associated with disease, drug resistance, multidrug resistance, herbicide resistance or not, insertions or deletions (indels) whether associated with a disease, the presence, absence, and/or abundance of viruses and viral variants, favorable or pathogenic bacterial, fungi, a non-invasive species, soil biome characterization (see if conducive/harmful to certain types of crops), and/or gut biome

The promiscuous probe may have a length that is between 20-35 bps without a locked nucleic acid or other melting temperature (Tm) increasing modification, or between 10-35 bps with a locked nucleic acid or other Tm increasing modification. The promiscuous probe optionally further comprising one or more of: between 35%-80% GC content; a Tm between 55° C.-62° C.; a binding site to the at least one nucleotide difference that is positioned either: in a middle region of the promiscuous probe length, wherein the middle region is defined in a central 50% portion of the probe length, or at an alternative location at least partially outside the middle region so long as promiscuous binding at a permissive temperature is maintained; and/or a locked nucleic acid at the at least one nucleotide difference.

The promiscuous probe may have a higher binding efficiency to the target polynucleotide sequence with the at least one nucleotide difference or may have a higher binding efficiency to the target polynucleotide sequence without the at least one nucleotide difference.

The method may use a plurality of promiscuous probes for multiplex detection of a plurality of nucleotide differences in the target polynucleotide sequence.

The promiscuous probe may be a fluorescent or fluorescently-labelled probe, including a labelled probe comprising a locked nucleic acid. The label may comprise a Taqman® label.

The promiscuous probe may have a greater than 98% binding region sequence complementary to a binding site of the target polynucleotide sequence for a high-hybridization efficiency condition, and less than 98% binding region sequence complementary to a binding site of the target polynucleotide sequence for a lower-hybridization efficiency condition.

The promiscuous probe may have a sequence configured to provide an at least 10% difference in amplitude of optical output for promiscuous probe bound to the first and second amplicons.

The method may further comprise the step of determining the permissive temperature by: contacting a positive control reaction mixture comprising an about 50:50 mixture of the first polynucleotide and the second polynucleotide with the primers and the promiscuous probe; and performing at least one PCR reaction on the control reaction mixture at a plurality of different temperatures spanning a low temperature that is below an optimal permissive temperature and a high temperature that is above the optimal permissive temperature; and identifying a temperature or temperature range in which both the first and the second polynucleotides are amplified and optically detected with a magnitude shift of fluorescent output between the first and second polynucleotides due to lower efficiency off-target binding of the promiscuous probe compared to higher efficiency on-target binding of the promiscuous probe. In this context, “about 50:50” reflects that the method can tolerate some variation in the precise ratio, including within plus/minus 20%.

The target polynucleotide sequence may be from a virus, including from a SARS-CoV-2 virus, and the at least one nucleotide sequence difference corresponds to a variant of the SARS-CoV-2 virus. This reflects that while the method is compatible with any of a range of applications, a particularly relevant application is to identify a virus variant. Optionally, the SARS-CoV-2 target polynucleotide sequence is from wild-type parental-Hu-1 (Accession NC_045512).

As described herein, the methods are useful for detecting any variant. For example, the variant may be a Variant of Concern 202012/01 of the lineage B.1.1.7; 20H/501Y.V2 of the lineage B.1.351; 20J/501Y.V3 of the SARS-CoV-2 P.1 lineage; 20C/S:452R; /6.1.429 of the lineage CAL.20C; a del69-70 mutation; a N501Y mutation; a E484K mutation; a E484Q mutation; a K417T mutation; a K417N mutation; a L452R mutation; a T478K mutation; a N679K mutation; or a Q954H mutation. The method and kit may be a permissive assay for the alpha (N501Y; HVdel69-70), beta (K417N; E484K), gamma (K417T;E484K), delta (L452R;T478K), epsilon (L452R), lambda (L452Q), mu (R346K), and omicron (N679K; Q954H) variants, for example.

The variant may comprise at least two mutations at different loci and the promiscuous probe may comprise at least two promiscuous probes each having a distinct fluorescence emission maximum. In this manner, the at least two mutations can be individually detected in a single well.

The probes and primers may comprise any one or more of the SEQ ID NOs provided herein (see, e.g., Table of Sequences appended herein), including SEQ ID Nos:1-15, and preferably comprises a forward primer, a reverse primer and a corresponding probe that targets a polynucleotide sequence between the forward and revers primers.

The method is compatible with a range of PCR assays, including a PCR reaction comprising digital PCR (dPCR) or droplet digital PCR (ddPCR).

The method of may have an at least 2-plex in one channel, with two channels in a single well, thereby providing a 4-plex per well. Of course, the methods are compatible with instruments having more than two channels.

The method may be configured for discriminating single-nucleotide polymorphism (SNP), cancerous mutation, pathological mutation, a deletion mutation, an insertion mutation, drug resistance mutation, multi-drug resistance mutation, herbicide mutation, multi-herbicide resistance mutation, reassortment mutation, or a biomarker mutation.

The method may be for a polynucleotide sequence obtained from organisms in wastewater.

The organism may be a virus in wastewater, the method further comprising the steps of: filtering the wastewater; concentrating the virus; extracting RNA; and determining a relative concentration of a wildtype virus and a variant virus in the wastewater. The methods and kits may be used with samples that may contain a mixture of WT and mutant polynucleotide.

In another embodiment, provided herein is a method of making an assay to detect a first polynucleotide target sequence having a variant sequence differing from a second polynucleotide target sequence by at least one nucleotide. The method may comprise the steps of: a) identifying the first and second polynucleotide target sequences; b) identifying an upstream flanking region and a downstream flanking region that are upstream and downstream from the polynucleotide target sequences; c) designing a forward primer and a reverse primer that specifically bind to the upstream and downstream flanking regions of the first and second polynucleotide sequences, with a separation distance between the upstream and downstream primer binding regions that is between 50 bps and 1500 bps, wherein the primers are configured to generate a first and a second amplicon product corresponding to at least a portion of the first polynucleotide target sequence and at least a portion of the second polynucleotide sequence; d) designing a promiscuous probe that will bind to the first and second amplicons with different hybridization efficiencies at a permissive temperature of between about 55° C. and 65° C.

The design of primers may be by selecting a flanking binding region of between 50 and 1500 nucleotides and the primer has at least 90%, at least 95% or 100% sequence complementarity to the flanking binding region.

The design of the discriminatory probe may comprise: performing a thermal gradient dPCR on a target mixture that comprising a mixture of the first polynucleotide target sequence and the second polynucleotide target sequence; selecting a temperature of maximal separation of output fluorescent amplitudes between the first and second polynucleotide target sequence amplicons in the target mixture, thereby identifying for any promiscuous probe the permissive temperature.

The method may further comprise the step of: performing the thermal gradient dPCR on: a first target that is 95%-100% a parent polynucleotide sequence; a second target that is 95%-100% the variant polynucleotide sequence.

The method may be for a greater than two-plex assay, wherein the positive control reaction mixture comprises a mixture of the greater than two polynucleotides. The method may be for a 5-plex 417/484 assay for a SARS-CoV-2 virus and variants thereof, wherein the positive control mixture comprises approximately equal concentrations of parental, alpha, beta, gamma, delta, epsilon, lambda, mu or omicron polynucleotide sequences corresponding to the S gene.

Also provided herein are kits for distinguishing a first target polynucleotide sequence from a second target polynucleotide sequence by dPCR or RT-qPCR. The RT-qPCR preferably uses the promiscuous probe in combination with a promiscuity-blocking nucleotide juror oligonucleotide (PBNJ) as described in U.S. Pat. App. No. 63/271,522 filed Oct. 25, 2021 (Atty Ref. 339033: 97-21P US). The first target polynucleotide sequence comprises at least one nucleotide difference not found in the second target polynucleotide sequence. The kit may comprise: for each location in the first target polynucleotide target sequence corresponding a nucleotide(s) difference: a set of PCR primers comprising a forward amplification primer and a reverse amplification primer which, when hybridized to the respective primer annealing sites, flank the location of the nucleotide(s) difference, wherein the set of PCR primers is capable of PCR amplification of corresponding regions of both the first polynucleotide target sequence and the second target polynucleotide sequence; a labeled promiscuous probe designed at a permissive temperature to hybridize at a first hybridization efficiency to the site of the at least one nucleotide difference of the first polynucleotide and at a second hybridization efficiency to the corresponding site lacking the at least nucleotide difference of the second polynucleotide, wherein the first hybridization efficiency is different than the second hybridization efficiency; a control mixture comprising a nucleic acid corresponding to the sequence of the first target polynucleotide; a control mixture comprising a nucleic acid corresponding to the sequence of the second target polynucleotide; and a control mixture comprising a nucleic acid mixture corresponding to the sequence of the first target polynucleotide and the sequence of the second target polynucleotide.

The kit may be for detecting a SARS-CoV-2 variant comprising primers and probes selected from the group consisting of: SEQ ID Nos: 1, 2, 3, 4, 5, and 7.

The kit may be for detecting an oncogene mutation, including in one or more of: BRAF600; TP53; EGFR, comprising one or more probes and primers selected from the group consisting of: SEQ ID Nos:16-27.

The kit may be for detecting a herbicide resistance mutation in a plant gene.

The invention may be a method, kit or composition of matter comprising any one or more of SEQ ID NOs:1-47, including groupings of respective primer pairs and corresponding probe, with multiplex applications comprising a plurality of such groupings.

### BRIEF DESCRIPTION OF THE DRAWINGS

FIGS. 1A-1C. Development of droplet digital PCR assays to distinguish parental-parental from del69-70 or N501Y SARS-CoV-2. Nucleic acids containing parental-parental or mutant S gene sequences were subjected to Bio-Rad's One-Step RT-ddPCR Advanced Kit or Probes. Parental or mutant populations were readily distinguishable by fluorescent amplitude. FIG. 1A depicts a robust linear dynamic range similar to the CDC N1 assay. FIG. 1B shows a simple linear regression analysis was performed using GraphPad Prism® software. FIG. 1C shows that while the CDC N1 primers and probe sequences were unable to distinguish B.1.1.7 synthetic RNA from RNA extracted from heat-inactivated parental virus (BEI NR-52286), the GT Molecular del69-70 or N501Y assays could delineate the two strain's sequences.

FIG. 2 shows the Detection of Wuhan, del69-70, and N501Y within a single well.

The ability of the assay to distinguish Parental from mutant S gene sequences was tested by generating a four-plex assay where ParentalN501 is detected at low amplitude in FAM, N501Y is detected at high amplitude in FAM, Parental HV 69-70 is detected at low amplitude in HEX, and del69-70 is detected at high amplitude in HEX. This technique affords four-plexing ability within a single well. See, e.g., FIG. 13, for a 2D plot.

FIG. 3 illustrates a Robust Linear Dynamic Range. To assess the linear dynamic range of a multiplexed assay for parental-parental and mutant S gene signatures, synthetic B117 SARS-CoV-2 RNA was first quantified and subjected to a 10-fold serial dilution curve and one-step RT-ddPCR. Simple linear regression analysis shows R² values>0.999. All data analysis were performed using GraphPad Prism.

FIG. 4 shows Detection of B.1.1.7 genetic signatures from United States wastewater. The ability of a four-plex assay was tested to differentiate Parental from del69-70 or N501Y SARS-CoV-2 in a municipality's composite wastewater sample. The assay was shown to detect the Parental strain S gene sequences at low amplitudes. The assay was also able to detect the presence of samples double positive for del69-70 (left; HEX) and N501Y (right; FAM). Based on this data a conclusion could be drawn that the virus shed in this municipality contains virus with features also present in hyper-transmissible SARS-CoV-2 strains.

FIGS. 5A and 5B show Robust Linear Dynamic Range. To assess the linear dynamic range of our GTM One-Step, synthetic B117 SARS-CoV-2 RNA was first quantified and subjected to a 10-fold serial dilution curve and one-step RT-ddPCR. Simple linear regression analysis shows R² values>0.999. All data analysis were performed using GraphPad Prism.

FIG. 6 shows that hyper transmissible strains from the B.1.1.7 lineage have a high amount of mutations shown here as the vertical lines on the grey bars which are representative of the viral genome. In one aspect, the test does not test for every mutation present in the alpha variant, instead 2 key mutations are targeted that have been previously described to have biological effects that drive the hyper-transmissibility of that alpha variant.

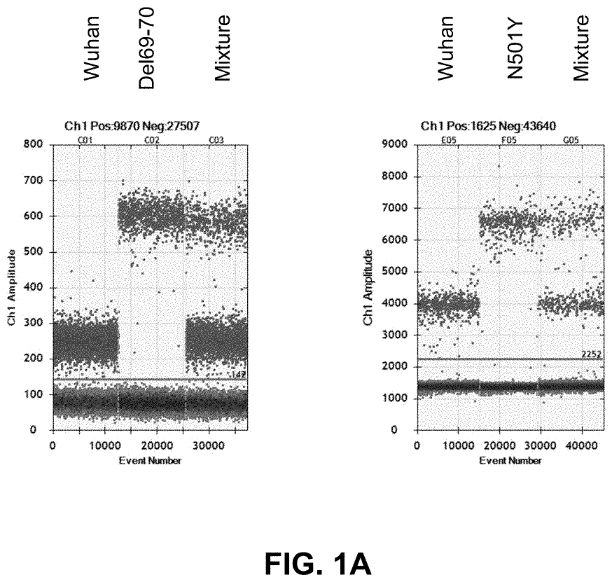

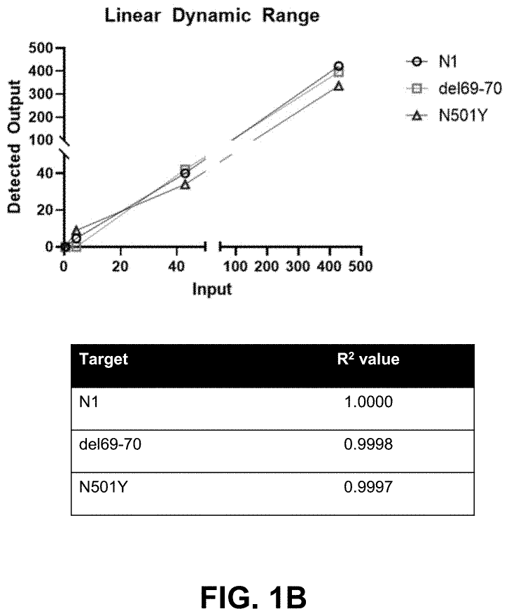

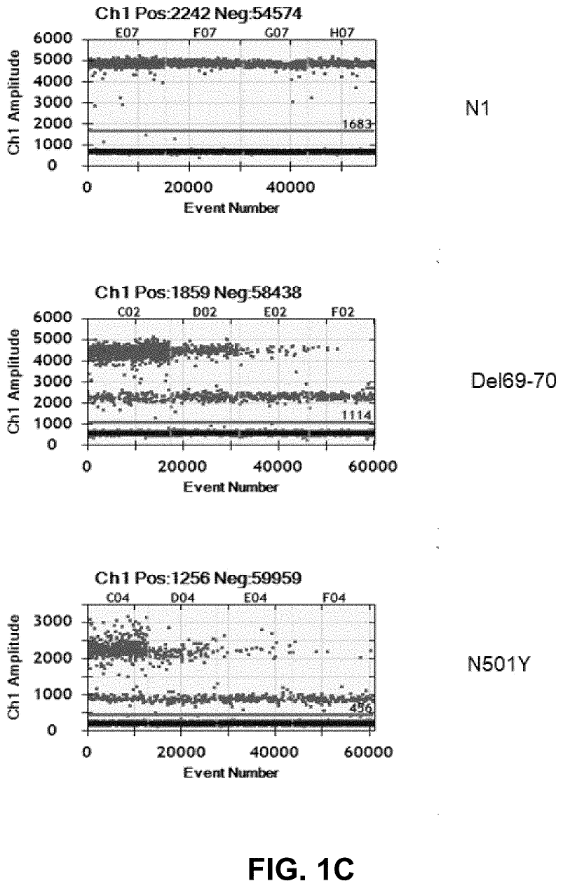

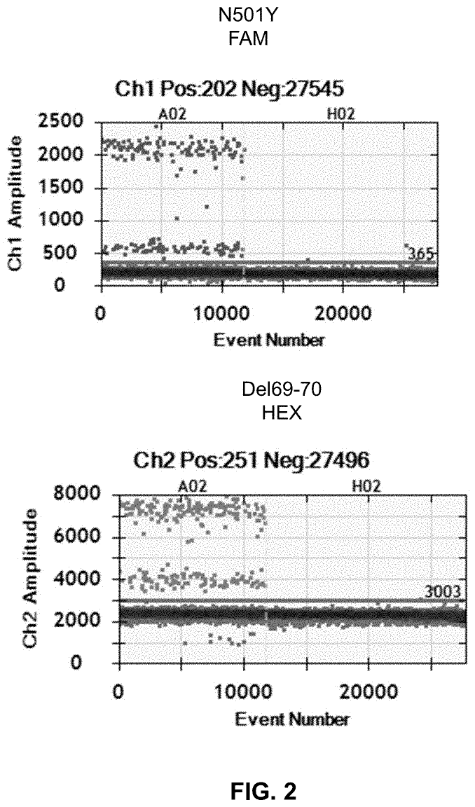

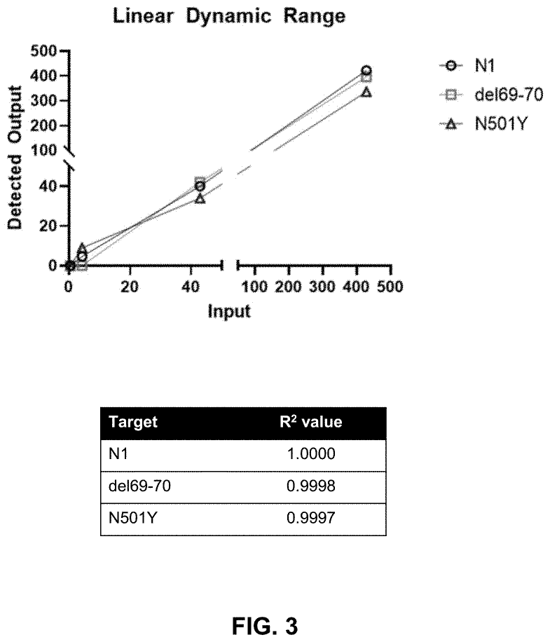

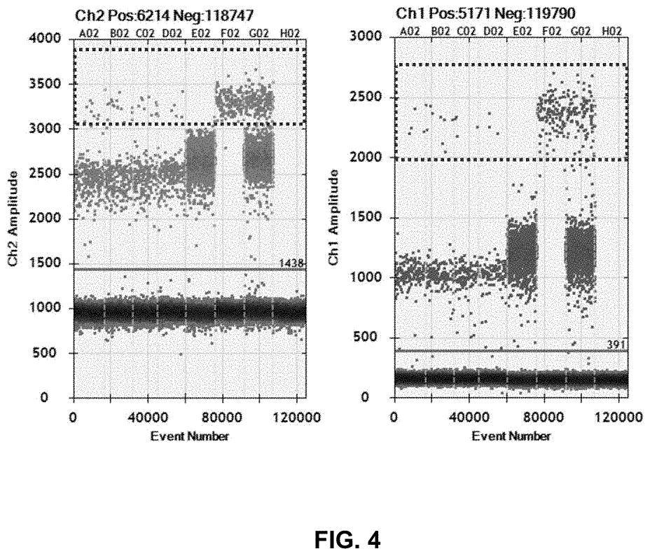

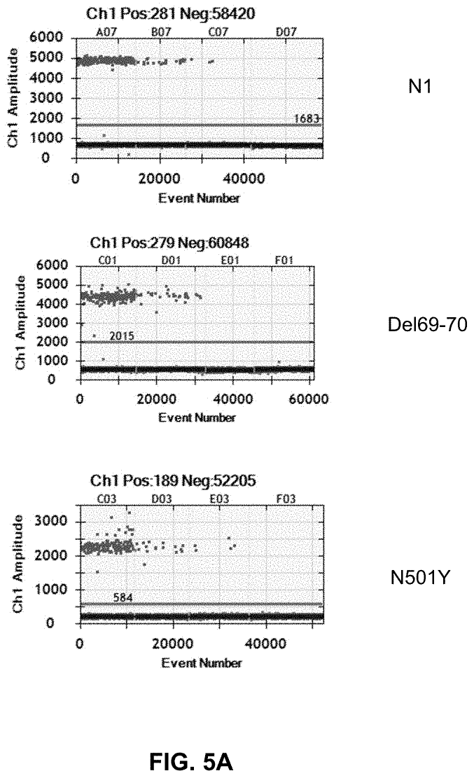

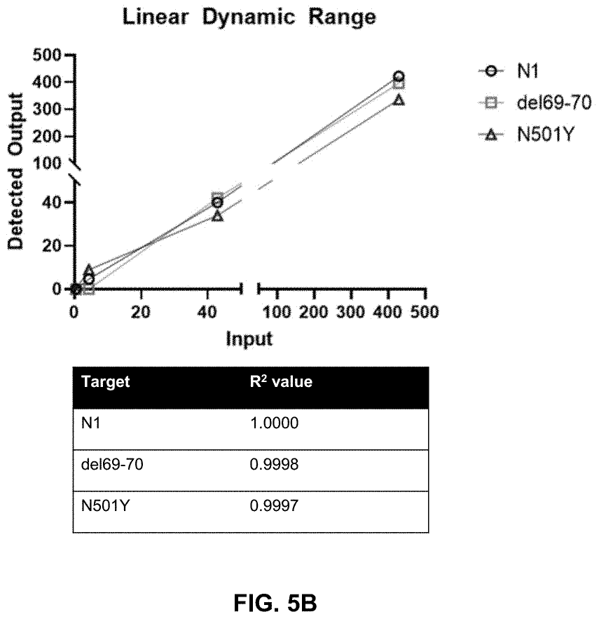

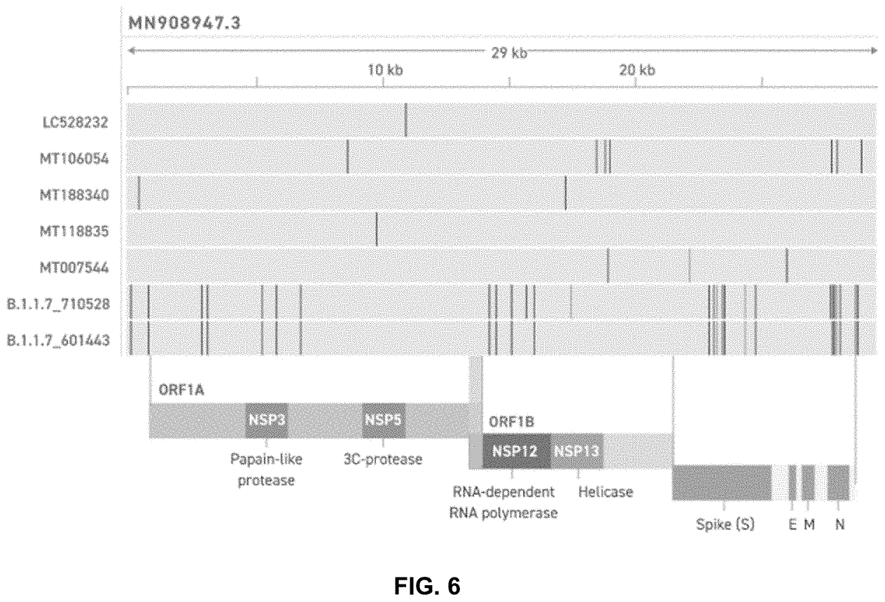

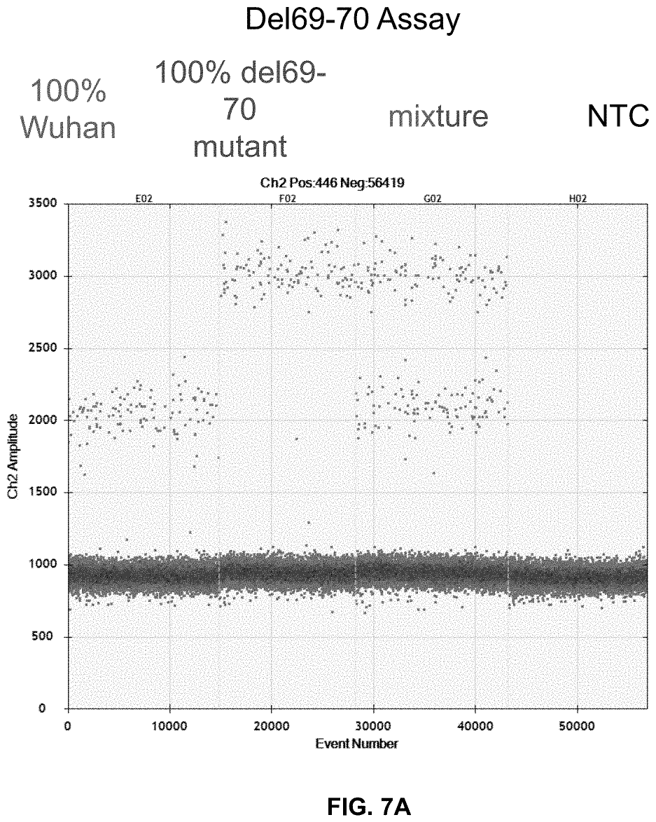

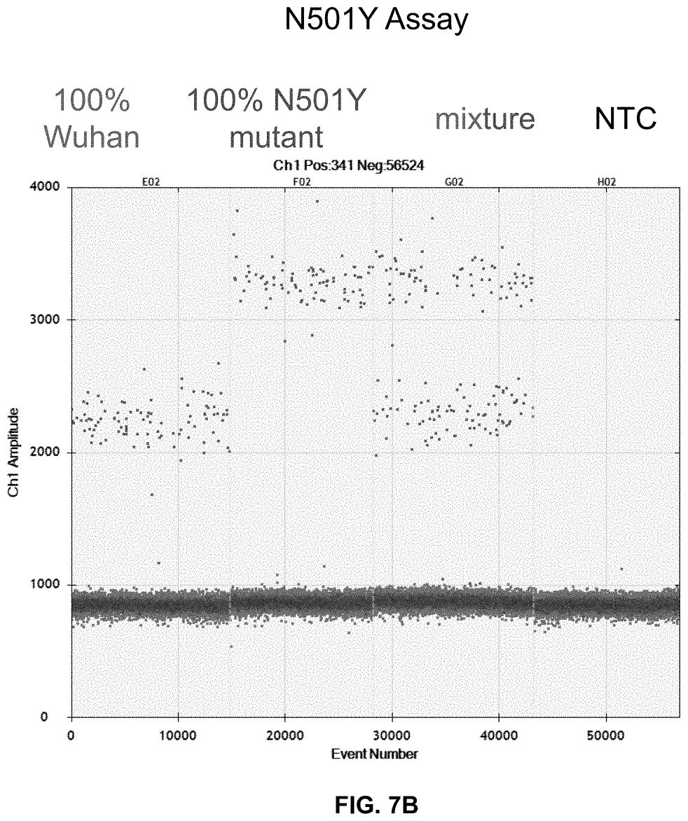

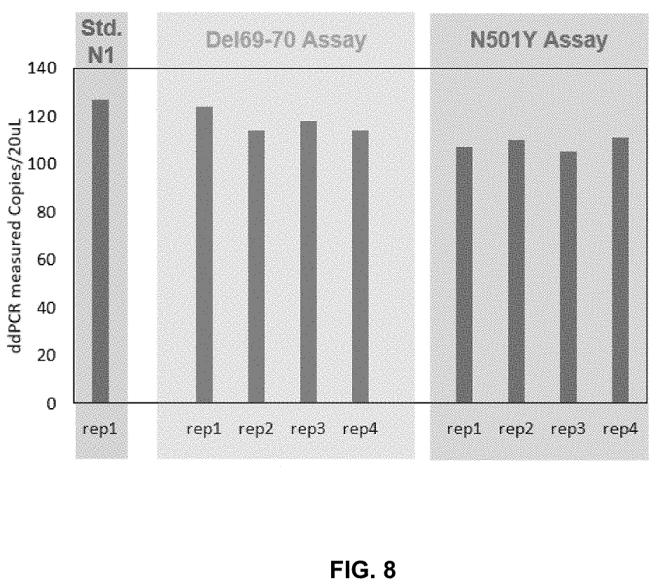

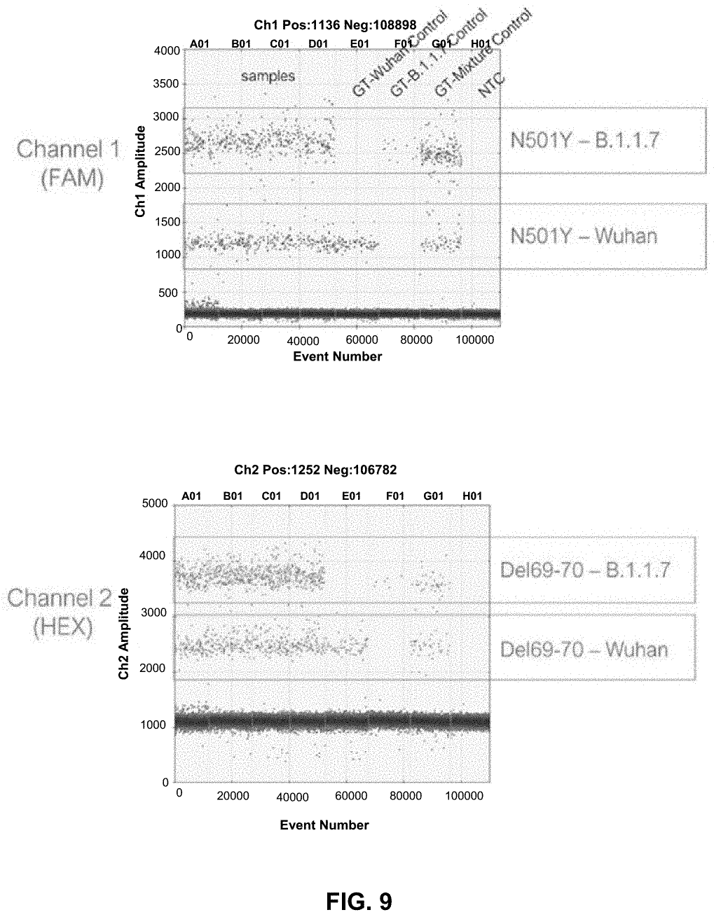

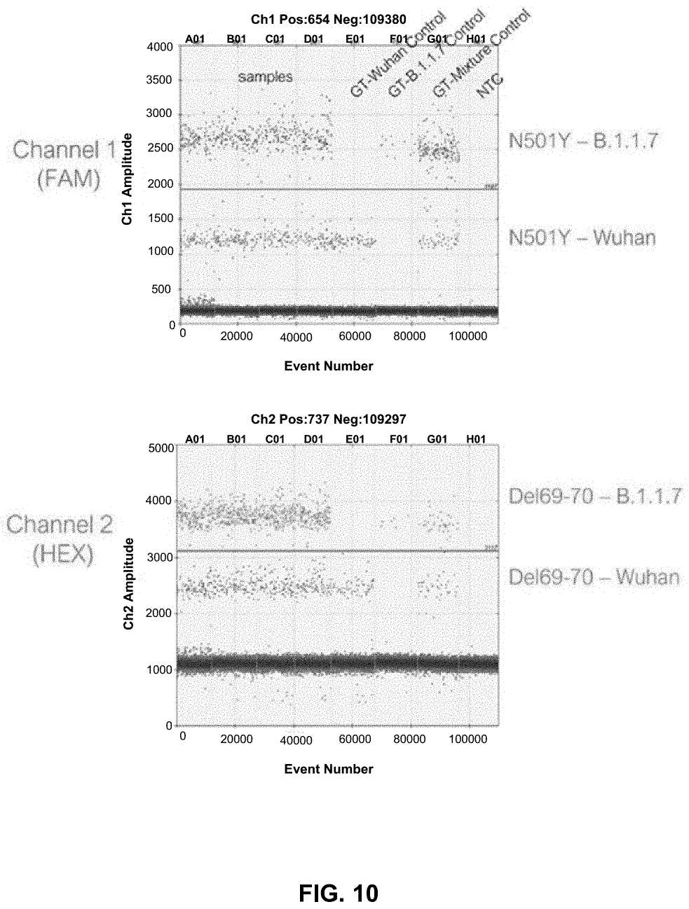

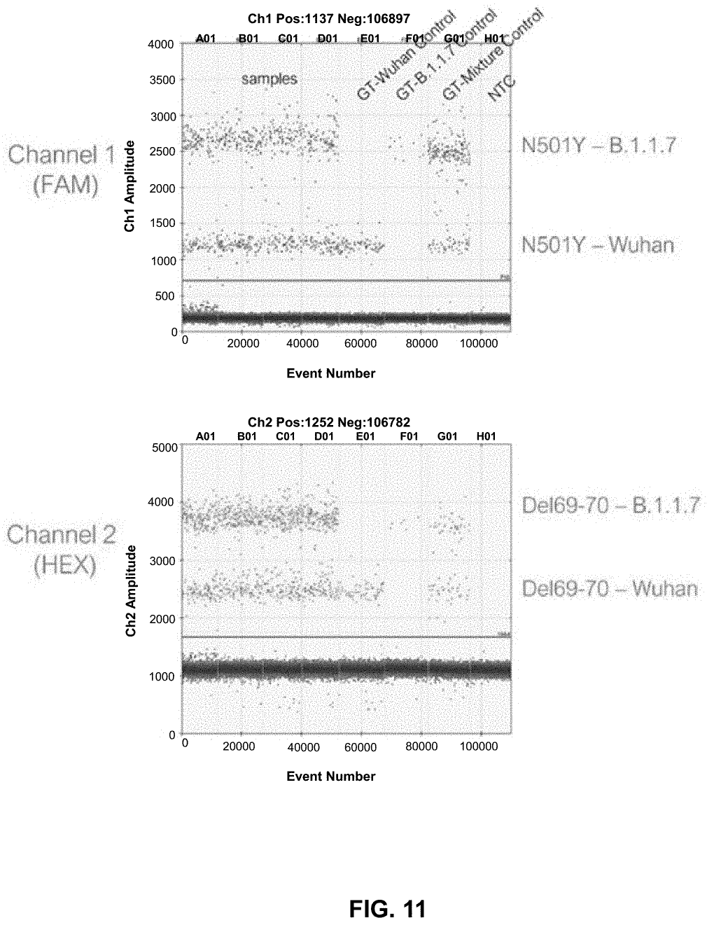

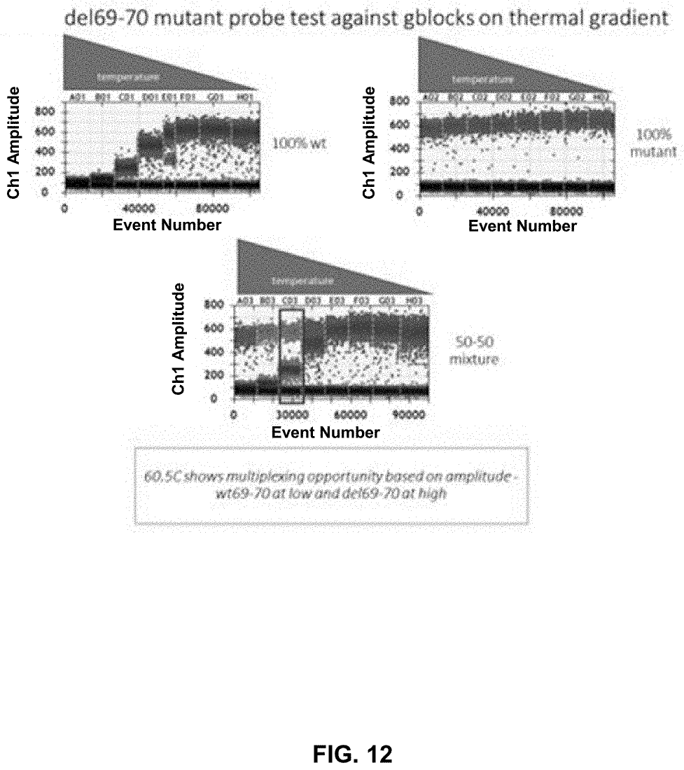

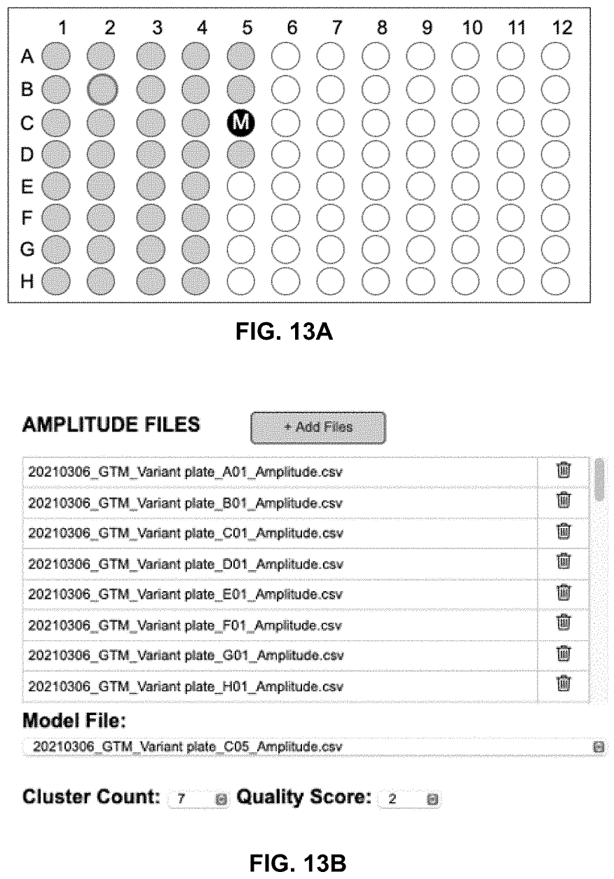

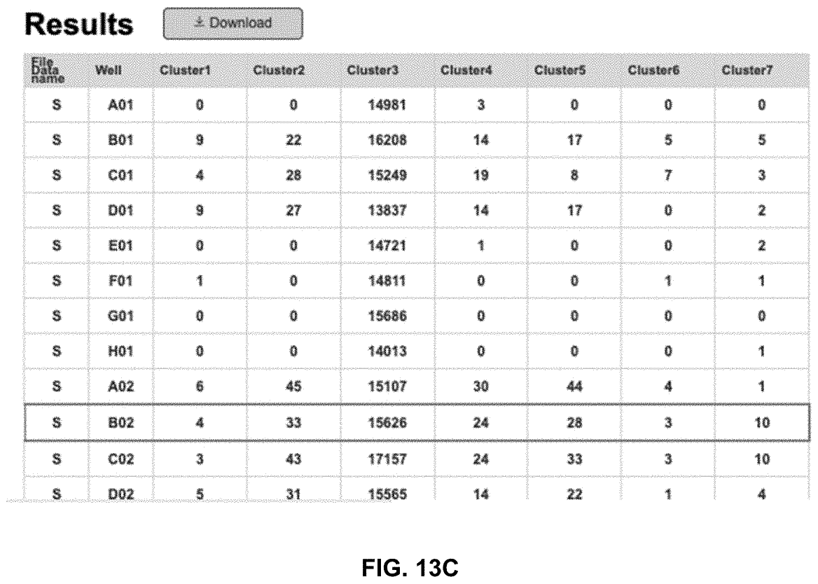

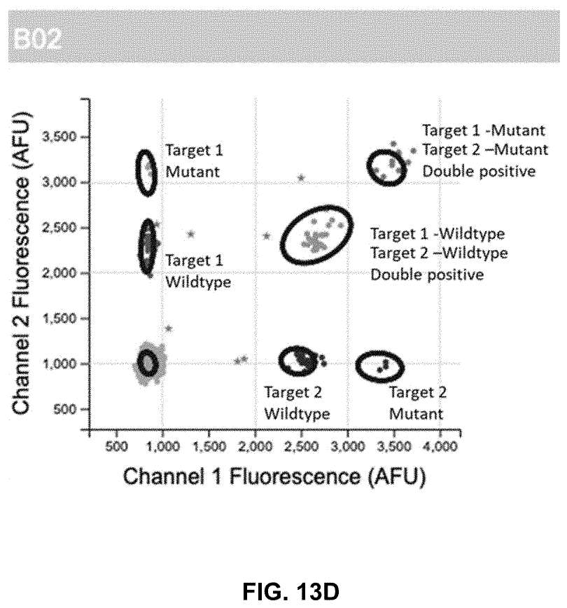

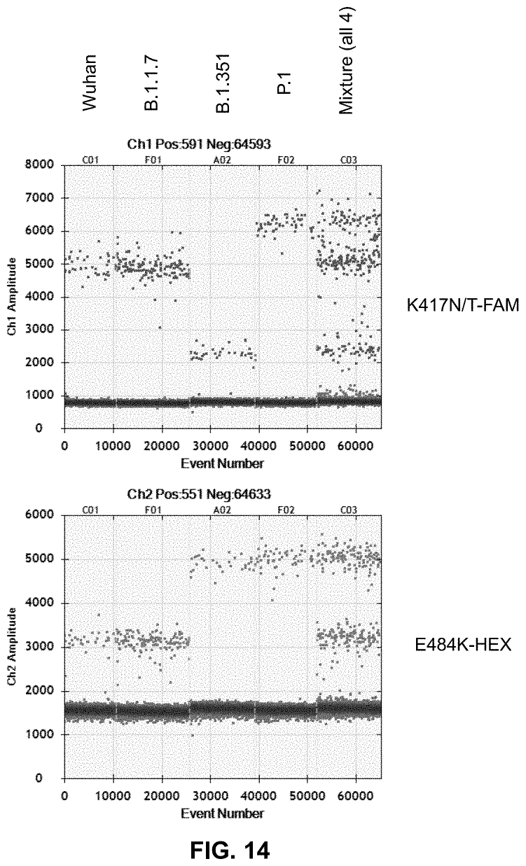

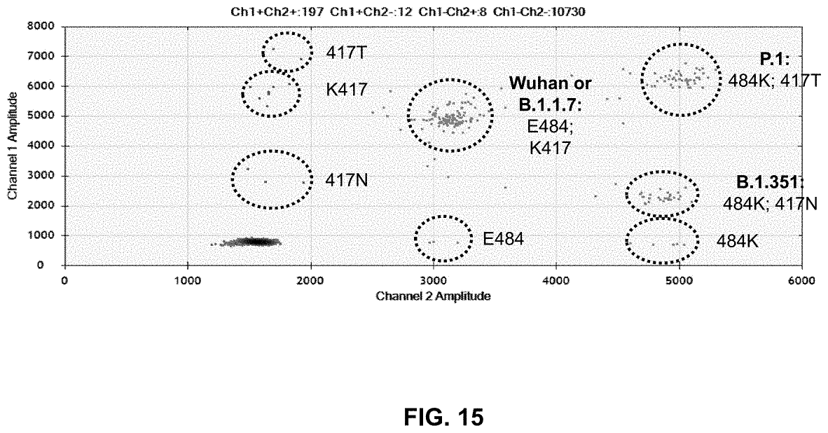

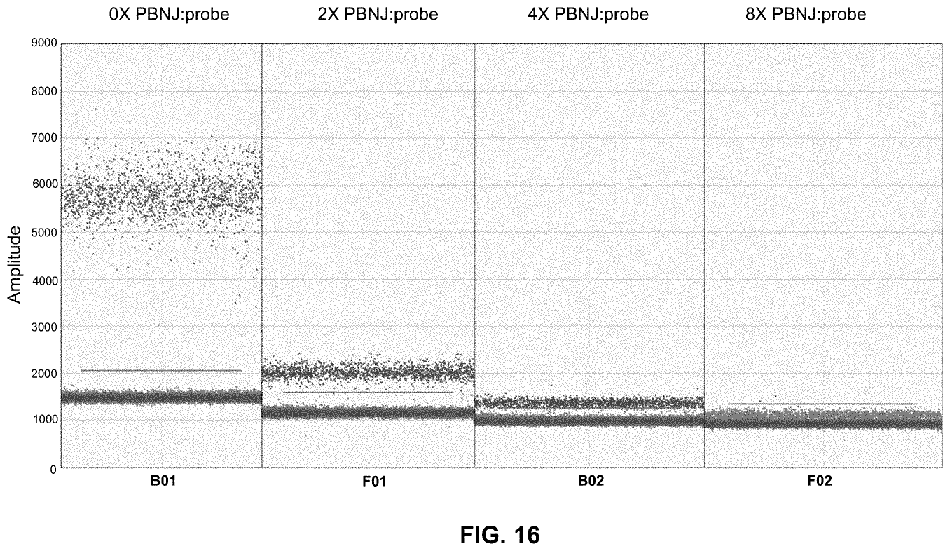

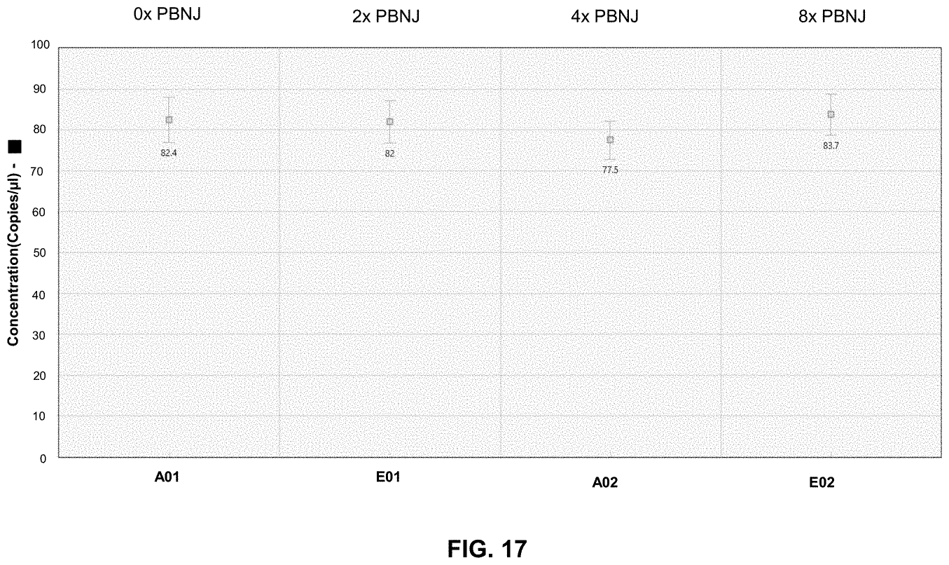

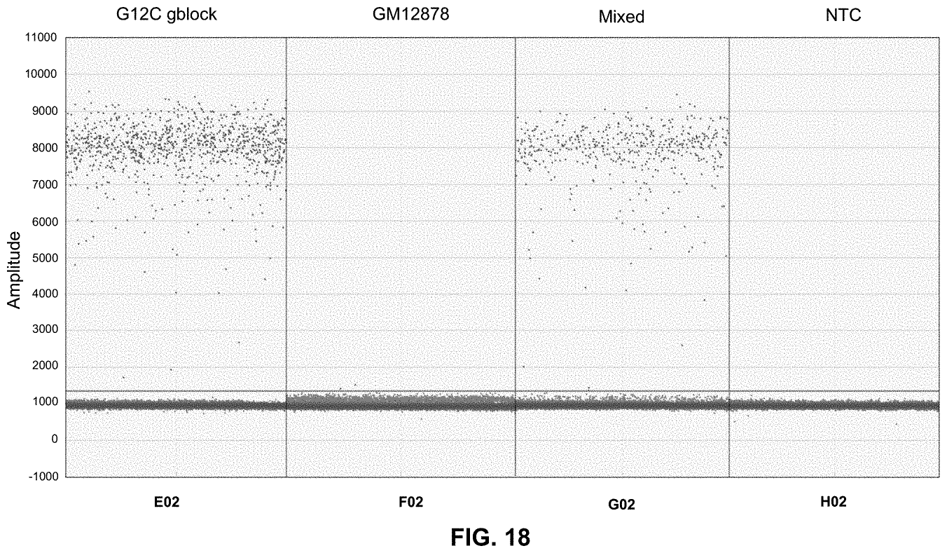

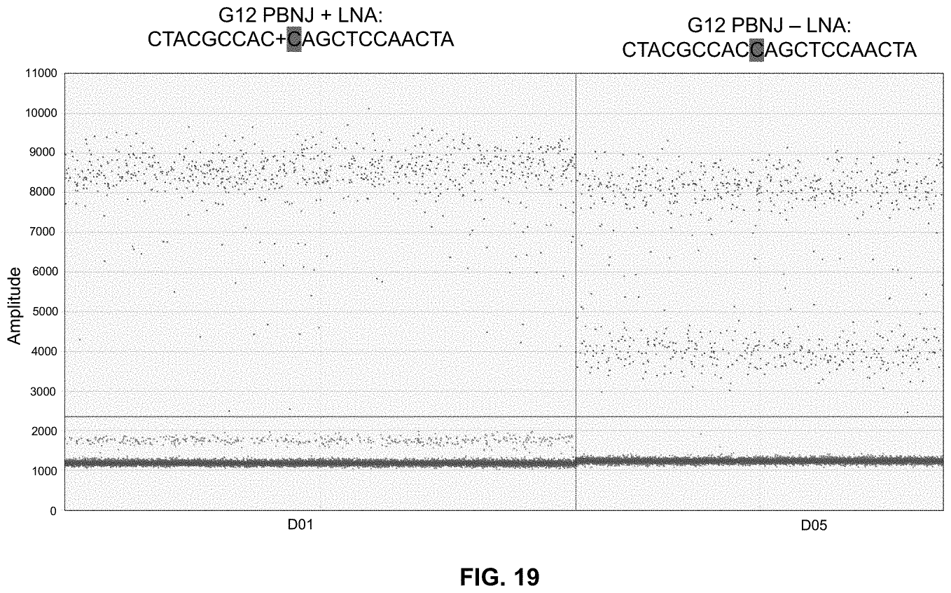

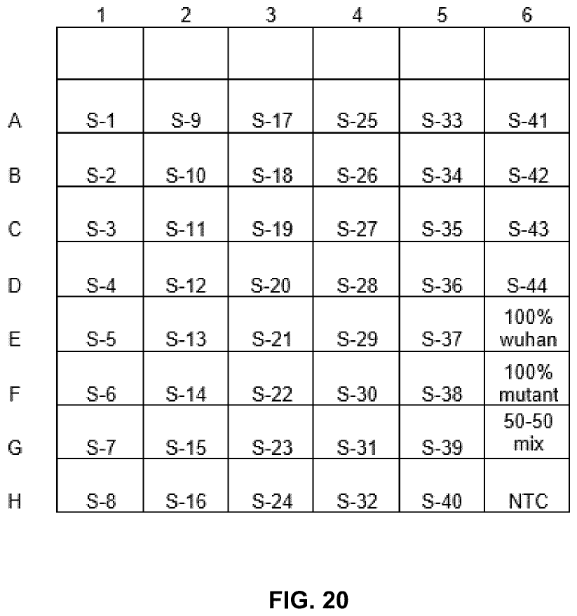

Both mutations have been found independently of one another. For example, the del69-70 mutations accounts for 2.5% of all sequences reported in the Europe. The N501Y mutation has been found in the beta variant of concern. The presence of both of these mutations together, however, is a strong indicator that the alpha variant or possibly another, related and yet to be defined hyper-transmissible variant is circulating within a community. The quantification of the N1 region is identical in all strains and does not differentiate. In one aspect, a variant wastewater test—quantifies two regions in the spike gene containing 2 key mutations known to drive hyper-transmissible function.

FIG. 7A shows the difference in amplitude of a promiscuous probe for the del69-70 mutation in a comparison of a polynucleotide comprising the del69-70 mutation to a polynucleotide from the parental SARS-CoV2 wild-type strain lacking the del69-70 mutation, along with a no-template control (NTC). FIG. 7B shows the difference in amplitude of a promiscuous probe for the N501Y mutation in a comparison of a polynucleotide comprising the N501Y mutation to a polynucleotide from the parental SARS-CoV2 wild-type strain lacking the N501Y mutation. These data reflect the methods provided herein are capable of distinguishing between target polynucleotides having at least one nucleotide variation with a single promiscuous probe.

FIG. 8 shows an accuracy determination comparing the N1 measured sequences to the spike gene sequences. Wastewater Sample 24 analyzed in classic ‘N1’ assay and found to have a concentration of 127 copies/20 uL well. The same extracted RNA from Sample 24 was analyzed for parental sequence at the locations of del69-70 and N501Y assay. Each test was run in replicate of 4. Results from an assay of the present disclose were within 7% and 14% of the validated N1 assay, respectively.

FIG. 9 shows a data plot for controls across two channels for the specified mutations.

FIG. 10 shows a data plot for controls across two channels and the specified mutations.

FIG. 11 shows a data plot for controls across two channels and the specified mutations.

FIG. 12 shows del69-70 mutant probe test against gblocks on thermal gradient.

FIGS. 13A-13D relate to a 2D plot of fluorescent intensity (amplitude) for two promiscuous probes, with one relevant to N501Y and another relevant for del69-70. FIG. 13A illustrates a 96-well plate set-up, with the optical output of well 2B displayed in FIG. 13D. FIG. 13B illustrates model files employed in software for the automated analysis. FIG. 13C are results of the analysis of various wells. FIG. 13D is a 2D plot of the two fluorescent channels, and corresponds to the row highlighted in FIG. 13C (e.g., Well B02). Use of two fluorescent labels having different fluorescent characteristics (such as emission wavelength) provides the ability to reliably detect relative amounts of wild-type, N501 mutant, Del69-70 mutant, and both mutations with only two probes. The relevant Cluster1-Cluster7 are identified in FIG. 13D. In this manner, quality-control is insured as well a demonstration of a 4-plex obtained in a single well for two promiscuous probes.

FIG. 14 shows the Detection of parental (wild-type), B.1.1.7, B.1.351, P.1 and a mixture of all 4 within a single well for K417N/T-FAM (top panel) and E484K-HEX (bottom panel) promiscuous probes.

FIG. 15 is a 2D plot of fluorescent intensity (amplitude) for the two promiscuous probes of FIG. 14, K417N/T and E484K. Use of two fluorescent labels (FAM and HEX) having different fluorescent characteristics (such as emission wavelength) provides the ability to reliably detect relative amounts of wild-type and various mutations, as annotated.

FIG. 16 is a plot of fluorescence intensity for various PBNJ:probe ratios (0, 2×, 4× and 8×), illustrating impact of PBNJ on nonspecific detection of KRAS 12C amplicon.

FIG. 17 is a plot of mutant concentration (copies/μL) for various PBNJ:probe ratios, illustrating PBNJ does not impact on-target detection.

FIG. 18 is a plot of fluorescent intensity for various targets with 8× PBNJ concentration, reflecting that 8× PBNJ completely removes nonspecific amplification.

FIG. 19 compares probe with (+) and without (−) locked nucleic acid (LNA) at the highlighted position. The LNA dramatically increases probe specificity and reflects the promiscuous probe is also useful for other applications, including oncogene applications.

FIG. 20 is a representative layout of a 48-well plate, including samples S-1 through S-44 and various controls at positions 6E-6H.

### DETAILED DESCRIPTION OF THE INVENTION

While the present disclosure may be applied in many different forms, for the purpose of promoting an understanding of the principles of the disclosure, reference will now be made to aspects illustrated in the drawings and specific language will be used to describe the same. It will nevertheless be understood that no limitation of the scope of the disclosure is thereby intended. Any alterations and further modifications of the described aspects, and any further applications of the principles of the disclosure as described herein are contemplated as would normally occur to one skilled in the art to which the disclosure relates.

As used herein “amplitude” refers to a magnitude or amplitude of fluorescence, also referred generally as fluorescence intensity. In certain aspects, amplitude may be used in reference to increased or decreased relative fluorescence.

As used herein “amplitude tuning” refers to harnessing that when a mis-match occurs within a probe binding region, a change in fluorescent amplitude can be detected due to a difference in hybridization efficiency. Probes are designed to have different hybridization efficiency between two amplicon sequences sharing less than 100% sequence identity. In certain aspects, the difference in hybridization efficiency is tuned to PCR parameters such as temperature and/or probe concentration. In this manner, a probe is characterized as promiscuous in that the probe is specifically configured to bind to both the “original” sequence and to the sequence sharing less than 100% sequence identity (also referred herein as having at least one nucleotide difference) at a permissive temperature, but at different hybridization efficiencies.

An “at least one nucleotide difference” refers to a pair of corresponding polynucleotide sequences that have at least one difference between two otherwise identical sequence regions. More specifically, the region corresponds to a region recognized by the probe, or the “probe recognition sequence”. The phrase at least one nucleotide difference is used broadly to encompass any type of difference, including by insertion, deletion, or a change in nucleotide. The difference may correspond to a single nucleotide difference or more than one nucleotide difference. The difference may be a contiguous difference in nucleotide differences. The difference may be at multiple distinct positions along the polynucleotide sequence. For convenience, the sequences may be referred to generally as a “target” or a “reference” polynucleotide sequence, from which that at least one nucleotide difference is desirably detected from a to-be-tested “test sample.”

As used herein, “Clustering” refers to a measure of fluorescence output for a target on a two channel output plot that is confined within a defined area, including an area distinct from another target. Accordingly, a “positive clustering” corresponds to an expected output plot that is clustered within an expected area. “Negative clustering” corresponds to output having a significant fraction of events that falls outside the expected area. Significant fraction may correspond to 10%, 5% or 1% of output falling outside the expected area. For example, the plot in FIG. 13D outlines the defined areas for each of four target polynucleotide sequences.

As used herein, “color channel” refers to a region of the light spectrum, including visible light, infrared light, and ultraviolet light. A color channel may be specified to be as broad a set of wavelengths or as narrow a set of wavelengths as useful to an individual practicing the methods disclosed herein. The color channel is generally matched to a probe label, such as the fluorescent emission wavelength of the probe label.

“Detecting” is used broadly herein to refer to methods that can identify and/or quantify within a target polynucleotide sequence. The methods herein preferably detect whether a difference is found from a sample and also quantify the differences. For example, for an application where the target polynucleotide is from a virus, one (e.g., a “first”) target polynucleotide sequence may correspond to a wild-type (e.g., “parental”) sequence, and another (e.g., a “second”) target polynucleotide sequence may correspond to a variant, wherein there is at least one mutation in the target polynucleotide sequence. Similarly, for a disease such as cancer, a first target polynucleotide sequence may correspond to a “normal” sequence and the second target polynucleotide sequence having a mutation that is associated with cancer. In this manner, the methods and kits are compatible with any polynucleotide sequence of interest that, with a change in sequence, there is an attendant impact or change in a state from a first state to a second state, including associated with function, a mutation conferring resistivity, disease, pathogenicity, risk factor, efficacy, diagnostic outcome, and the like.

As used herein, “permissive” refers to a condition, such as temperature, which allows hybridization of a probe to more than one polynucleotide sequence, but at different hybridization efficiency, thereby accommodating at least one mismatch.

As used herein, “probe” refers to a labeled oligonucleotide designed to be at least partially complimentary to a target DNA sequence of interest such that when combined with a hybridization reaction it can bind to and detect the target. A probe may have more than one possible hybridization target and depending on reaction conditions, e.g. temperature, may bind to only one target, two targets with different hybridization efficiency or no targets.

As used herein, “promiscuous probe” refers to a probe which hybridizes to more than one polynucleotide sequence at a permissive temperature. In certain aspects, a promiscuous probe can hybridize to a polynucleotide despite one or more than one sequence mismatch. Of courses, exact 100% match provides a higher hybridization efficiency than less than 100% match. Generally, the more percentage mismatch, the lower the hybridization efficiency. In certain aspects, a promiscuous probe hybridizes to two or more polynucleotides with less than 100% sequence identity and the hybridization efficiency of the promiscuous probe is different between the two or more polynucleotides depending on the base sequence of the polynucleotides. Furthermore, temperature is an independent parameter for also affecting hybridization, with higher temperatures requiring higher sequence matching for hybridization. The permissive temperature can be selected from a range of permissive temperatures. In this manner, the permissive temperature used in the method may be optimized so as to maximize fluorescent intensity output differences between on- and off-target binding.

The promiscuous probe, as desired, may comprise a locked nucleic acid (LNA). A LNA has a chemical modification, such as an extra bridge associated with the ring structure (including between the 2′-O and 4′-C carbon atoms), that effectively locks the conformation and generally provides increased stability against enzymatic degradation, and improves specificity and affinity of the probe (as reflected by an increase in Tm by about 2° C.-4° C. per LNA. This provides another means of adjustment of probe hybridization properties to ensure appropriate affinity characteristics to a target (wild-type) and the off-target (e.g., mutation) at an appropriate temperature range.

As used herein, “PCR” or “Polymerase chain reaction” refers to the well-known technique of enzymatic replication of nucleic acids which uses thermal cycling for example to denature, extend and anneal the nucleic acids.

As used herein, “Sample polynucleotide” refers to a biological sample having a target polynucleotide sequence that is detected, including without and/or with the difference in target polynucleotide sequence. For example, the sample may comprise a mixture of polynucleotides containing different sequences at the loci of interest.

As used herein, “Target polynucleotide of interest” refers to a portion of a longer polynucleotide, including a portion that may or may not contain a relevant at least one nucleotide difference. The polynucleotide may comprise RNA or DNA. Optionally, RT-PCR may be performed on the RNA to generate DNA that is then subject to PCR.

As used herein, “Target threshold” refers to use of a PCR reaction on a positive control reaction mixture to identify fluorescent amplitude output ranges associated with the respect target polynucleotide sequence. This thresholding aspect may be implemented in software that is used to identify and quantify target polynucleotide sequences, including a plurality of targets. The implementation may be in terms of creating a model file with attendant edge definition, such as an edge shape that is ellipsoid to include all relevant target within the edges. In this manner, automatic thresholding of signals/results for each target is obtained. See, e.g., FIG. 13D.

Probes may be of any functional length. Without limitation to any particular embodiment, probes may be of 10 to 100 nucleotides in length, 15 to 90 nucleotides in length, 25 to 75 nucleotides in length, 30 to 50 nucleotides in length, 37 to 43 nucleotides in length or any combination thereof.

Probes may be labeled by any means known in the art. The label on the probes may be fluorescent. The light emitted by the label on the probes may be detectable in the visible light spectrum, in the infra-red light spectrum, in the ultra-violet light spectrum, or any combination thereof.

In certain aspects, a promiscuous probe has a length that is between 20-35 bps without a locked nucleic acid or other melting temperature (Tₘ) increasing modification, or between 10-35 bps with a locked nucleic acid or other Tₘ increasing modification; the promiscuous probe may optionally be further characterized by one or more of a GC content between 35%-80% and a Tₘ between 57° C.-62° C.:

In certain aspects, a promiscuous probe has greater than 98% binding region sequence complementary to a binding site of the target polynucleotide sequence for a high-hybridization efficiency condition, and less than 98% binding region sequence complementary to a binding site of the target polynucleotide sequence for a lower-hybridization efficiency condition.

Any of the methods and kits may be used with a PBNJ, including for RT-qPCT applications. The PBNJ may contain a locked nucleic acid (LNA) at a SNP position. The PBNJ has a reference binding region and an extension blocker that prevents elongation by a polymerase. In this manner, the PBNJ at a competitive concentration relative to the promiscuous probe suppresses promiscuous probe binding to one the sequences with amplification to further discriminate the sequences via PCR.

In certain aspects, the presently disclosed methods and kits can be used for discriminating single-nucleotide polymorphism (SNP), cancerous mutation, pathological mutation, a deletion mutation, an insertion mutation, drug resistance mutation, multi-drug resistance mutation, herbicide mutation, multi-herbicide resistance mutation, reassortment mutation, or a biomarker mutation.

The present disclosure describes methods and kits for the targeted collection of polynucleotide sequence information. In certain aspects, methods and kits are disclosed wherein labeled probes provide for the detection of the presence or absence of at least one nucleotide difference between a first polynucleotide sequence and a second polynucleotide sequence. In certain aspects, methods and kits disclosed herein provide for multiplexing of molecular assays which result in efficiencies in the utilization of testing supplies, such as PCR reagents, as well as containers in which PCR reactions are run.

The methods and kits provided herein are useful for detecting and quantifying a sequence difference relative to a reference polynucleotide sequence. Generally, target polynucleotide sequences are examined to determine if any of the sequences vary by an at least one nucleotide difference. The methods provided herein can, as desired, detect and quantify these differences. This can be of particular use when the target polynucleotide sequence is from a virus, because viruses can have relatively high rate of mutation. In such a situation, the at least one nucleotide difference may correspond to a variant, and it is desirable to quantify the amount of variant relative to a parent or wild-type target polynucleotide sequence.

In certain aspects, the need for fluorescently labeled probes is reduced by up to 67% or half in comparison to common utilized molecular assays. In one aspect, the reduction in assay required probes is achieved by application of amplitude tuning which can be achieved through, primer and probe design with respect to hybridization targets. In some aspects, when a mis-match occurs between target and the probe, that mis-match can be assessed in terms of a fluorescent amplitude change from an output signal. PCR parameters can be fine-tuned such that at a specified permissive temperature one probe is capable of recognizing both on and off-target nucleic acids.

In some aspects, the ability to provide individual probes for what may be considered multiple hybridization targets (e.g., “promiscuous probes”), albeit of different hybridization efficiencies, provides for multiplexing of reactions. In one aspect, a 4-Plex dPCR method is enabled to run four targets in one well of a dPCR plate instead of two wells (two targets in each well). Depending on the number of targets a promiscuous probe is able to quantify, there is significant savings associate with reagent costs and decrease in the number of wells. Accordingly, relevant features and benefits of the methods, kits and assays, include, savings of up to 67% on all reagent costs which are a significant part of the operational costs of digital droplet PCR; use up to one-third of the wells so one can run more samples through one machine, thereby doubling or tripling output without having to purchase an additional ddPCR machine; rapid assay development because one probe per well may recognize up to three targets, instead of having three probes per well for three targets.

Samples of polynucleotides which can be analyzed by the presently disclosed methods and kits can be derived from any source comprising a polynucleotide of interest. Possible sources of samples include but are not limited to an organism selected from the group consisting of a virus, a bacteria, a fungus, a parasite, a plant cell, an animal cell, or a cancer cell. Further, possible sources of samples include but are not limited to an environmental sample, soil, seed, plant material, wastewater sample, industrial water sample, natural water sample (including river, lake, stream, ocean, groundwater, well water, aquifer), a biological sample such as a gut/stool sample, a liquid or tumor biopsy from a cancer patient, a swab or saliva sample, an animal sample (veterinary/animal husbandry).

In certain aspects, a test sample is analyzed for short or single nucleotide polymorphisms (SNPs) whether associated with disease, drug resistance, multidrug resistance, herbicide resistance or not, insertions or deletions (indels) whether associated with a disease, the presence, absence, and/or abundance of viruses and viral variants, favorable or pathogenic bacterial, fungi, a non-invasive species, soil biome characterization (see if conducive/harmful to certain types of crops), and/or gut biome.

Kits are disclosed herein, including for providing a PCR multiplexing approach—based on PCR efficiency, including but not limited to dPCR and ddPCR. In certain aspects, the presently disclosed kits provide for a 4-plex in one channel with two probes, thus saving 50% in reagents used in conventional assays having one fluorescent probe type per individual nucleotide sequence of interest.

In certain aspects, a kit comprises validated RNA or DNA standards, primer/probe mix, and validated conditions. The kit may contain the standard PCR reagents, including enzymes (DNA polymerase), deoxynucleotide triphosphates (dNTPs) and PCR buffers.

### EXAMPLES

The following examples are presented in order to more fully illustrate some embodiments of the invention. They should in no way be construed, however, as limiting the broad scope of the invention. Those of ordinary skill in the art can readily adopt the underlying principles of this discovery to design various compounds without departing from the spirit of the current invention.

### Example 1

### Probe and Primers

Probes were designed to contain a viral mutation of interest at the center of the probe. The ends of the probe on either side of center may then have sequences corresponding to the polynucleotide sequence, which may be identical for both polynucleotide sequences (e.g., parent v. variant; normal v. mutant; etc.). Primers were designed to flank the region that is targeted by the probe. Primer and probe solutions were formulated at either 500 nM primers/125 nM probes or 900 nM primers/250 nM probes. DNA or RNA control template containing sequences of interest was utilized comprising an equal mixture of wildtype and mutant sequences and a thermal gradient ddPCR experiment was performed spanning strict temperatures (high temperatures that drive specificity and thus amplification on mutant templates is seen) to permissive temperatures (low temperatures in which the probe that is specific to mutant sequence also binds the wildtype sequence). A temperature was identified in which both targets (wildtype and mutant) were amplified. Because ‘off target’ probe binding (in the case of the wildtype sequence) causes the reactions to be less efficient, the amplitude was shifted down allowing for differentiation of wildtype and mutant(s).

For each mutation of interest, ‘permissive’ temperatures are identified that allow multiplexing the wildtype and mutant targets using a single probe. To be able to multiplex 2 different genome locations (thus four targets total) temperatures and tune melting temperatures are identified such that the probes are each ‘promiscuous’ at the same temperature. As desired, LNA can be added, including to the N501Y probe, thereby ensuring the probe is permissive for both wildtype and mutant at the same temperature as another probe, including a del69-70 probe.

### Example 2

### Wastewater Test Kit

In one aspect, the assays of the present disclosure have been developed in the form of the GT RT-ddPCR (Parental+B.1.1.7) 4-plex wastewater test kit which is a molecular reagent kit containing all primers, probes, and controls for absolute quantification of strains containing both the Parental sequence and the B.1.1.7 mutated sequences at two viral genome locations, those coding for the del69-70 and N501Y mutations. This embodiment targets 2 key mutations that have been previously described to have biological effects that drive the hyper-transmissibility of the lineage. Both mutations have been found independently of one another. For example, the del69-70 mutations accounts for 2.5% of all sequences reported in the Europe2. The N501Y mutation has been found in the beta, gamma, and mu variants of concern, interest, or being monitored. The presence of both mutations together, however, is a strong indicator that the alpha variant or possibly another, related and yet to be defined hyper-transmissible variant is circulating within a community.

The user of the wastewater test kit can utilize only two wells with only two fluorophores/probes per well to be able to test for a WT and two other mutations in the WT.

Many conventional assays require the use of more probes and reagents and two wells in a plate for each sample. In some aspects, the presently disclosed methods save up to 67% on all reagent costs which are a significant part of the operational costs of PCR, e.g. droplet PCR. Further the presently disclosed methods all usage of just half of the wells of conventional techniques so that one can run more samples through one machine, thereby doubling output.

The presently disclosed assay can also promote rapid assay development because only one probe per well can recognize two or more targets, instead of convention methods requiring two probes per well for two targets.

The presently disclosed methods also reduce multiple targets effects due to non-specific amplification due to very minor changes between nucleic acids. The present methods harness the off-target amplification, albeit at less efficiency, to discriminate these nucleotide mismatches. The presently disclosed methods can also be used in contexts, including but not limited to, multiplexing liquid biopsy assays (for cancer and others), custom genotyping assays, and viral mutation assays.

### Example 3

### Detection of SARS-CoV-2 B.1.1.7 Lineage Signatures in Wastewater

Wastewater pathogen monitoring provides community-wide surveillance to estimate disease prevalence and tracking. This surveillance paradigm affords the opportunity to assess viral load within a community without the need for large-scale diagnostic testing. Further, monitoring viral load over time provides communities with actionable data for local economy decision making. Several industry and academic groups successfully implemented and deployed SARS-CoV-2 wastewater monitoring services as a tool to combat the COVID-19 pandemic.

Wastewater pathogen monitoring provides various levels of resolution. For example, samples collected from individual dormitories identify localized outbreaks and thus highly tuned quarantine strategies. At a larger scale, testing samples from wastewater treatment facilities allows monitoring communities larger scale disease outbreaks.

A SARS-CoV-2 wastewater monitoring service was expanded to include a molecular assay targeting mutations found in the hyper-transmissible B.1.1.7/alpha variant strain. SARS-CoV-2 entry into human cells is dictated by interactions between the viral Spike protein and the human ACE2 protein. The B.1.1.7 strain contains six mutations within the Spike protein, thereby hinting these mutations may render a virus that either a) evades immune system recognition or b) has a higher affinity for ACE2. While testing a sample for the presence of all 6 mutations is complex, costly, and potentially redundant, we narrowed our strategy to include two mutations likely involved in promoting hyper-transmissibility.

Lessons learned from the Denmark mink outbreak helped inform testing strategy. Between June and November 2020, SARS-CoV-2 spread to over 200 mink farms and ultimately led to the culling of all Danish mink herds. We noted two similar mutations between the mink strain and B1.1.7: deletion of HV residues at positions 69-70 of the Spike protein, as well as an amino acid substitution within the receptor binding domain at residue 501. Further, the recent beta and gamma variant strain thought to emerge independently of the alpha Variant strain also contains the N501Y mutation. It was postulated that the independent emergence of these mutations indicate selective advantage that render hyper-transmissibility. Thus, we selected del69-70 and N501Y as our targets.

Presently described is a novel multiplexed design strategy to detect both parental and B.1.1.7 signatures, namely del69-70 and N501Y, within a single digital PCR well. This approach was coupled the molecular tools provided by the permissive probes with a SARS-CoV-2 wastewater testing service and detected these two mutations in United States wastewater samples obtained from a wastewater treatment facility serving a community known to contain the alpha Variant.

The presently disclosed molecular tool can in some aspects be used to monitor viral loads over time to determine if wastewater monitoring mirrors hyper-transmissibility seen within a community. Monitoring wastewater for these mutations will ultimately provide a multi-disciplinary approach to translate molecular biology to sewer genotyping to epidemiology.

COVID and COVID variants: B1.1.7 (alpha varian) mutations—all in spike gene; South African (beta variant)—all in spike gene; E484K; K417N.

This ddPCR method in which we obtain a readout on both mutant and wildtype sequences using a single probes may be used for any genotyping tests in which ultrasensitivity is required. Examples include, but are not limited to, circulating tumor DNA, viral mutants, non-invasive prenatal testing.

Find example genome sequences that have the mutation of interest, align with parental sequence, find flanking regions around mutation, design primers that will flank the mutation and design probe to either parental or mutant sequence. Try to get the mutation in the perfect center of the probe. Target Tms around 60 degrees. Once we have primers and probes, formulate to 22× such that our final standard concentrations are achieved adding 1 uL (500 nM primers, 125 nM probe; or 900 nM primers, 250 nM probe). Perform a thermal gradient on three different targets, a target that is 100% parent/wt/Wuhan, a target that is 100% mutant/alpha/B.1.1.7 in this case, and a target that is a 50-50 mixture of the two.

Select condition in which we see maximal separation of amplitudes for mutant and parental and negative droplet populations in the 50-50 mix control. This condition also must be specific for both parent and mutant sequences and thus the parent/wt population is absent in the 100% mutant and mutant population is absent in the 100% mutant sample.

### Example 4

### Storage, Stability and Methodology

Store at −20° C. and minimize freeze/thaw cycles. The Controls are composed of DNA or RNA. Aliquoting and refreezing solution to avoid freeze/thaws is highly recommended.

The GT-ddPCR Mutation Detection Assay comprises pre-mixed primer probe solution for 4-plex quantification of parental and variant viral sequences and appropriate controls. Each kit comes with 200 μL of the 20× assay mix sufficient for 200×20 μL reactions.

TABLE 1
Kit Contents

| | Qty | Volume | Storage |
|---|---|---|---|
| Reagent | (Tube) | (uL) | (° C.) |
| GT-4plex Primer-Probe | 1 | 200 | −20 |
| Solution | | | |
| GT - Variant Positive | 1 | 100 | −20 |
| Control | | | |
| GT- parental Positive | 1 | 100 | −20 |
| Control | | | |
| GT-Mixture-Control | 1 | 100 | −20 |

### Reagents and Equipment

1-Step RT-ddPCR Advanced Kit for Probes (Bio-Rad catalog #1864021); QX100™ or QX200™ Droplet Generator (Bio-Rad catalog #1863002 or 1864002, respectively) or Automated Droplet Generator (catalog #1864101); QX100 or QX200 Droplet Reader (Bio-Rad catalog #1863003 or 1864003, respectively); C1000 Touch™ Thermal Cycler with 96-Deep Well Reaction Module (Bio-Rad catalog #1851197); PX1™ PCR Plate Sealer (Bio-Rad catalog #1814000).

Reagents for RNA Purification: The QIAGEN QlAamp Viral Mini Kit (Catalog #52906, #52904) is validated for use with all GTddPCR Mutation Detection Assays per the manufacturer's instructions. Alternative isolation kits that generate high quality, purified RNA are likely compatible.

TABLE 2
GT-ddPCR (parental + B.1.1.7) 4-plex Wastewater Test Kit Workflow
Workflow

| | |
|---|---|
| Step 1 | Isolation of viral RNA |
| Step 2 | Prepare PCR-Ready Samples |
| Step 3 | Generate Droplets |
| Step 4 | Perform PCR |
| Step 5 | Read Droplets |
| Step 6 | Analyze Results |

TABLE 3
General Laboratory Equipment (Not Provided)

| Description | Source |
|---|---|
| Single and multichannel adjustable pipettors | Rainin or Eppendorf |
| (1.00 μL to 1,000 μL) | |
| Microcentrifuge | Multiple Suppliers |
| Microwell plate centrifuge, with a rotor that | Multiple Suppliers |
| accommodates standard microplates | |
| Laboratory freezers −20° C. and −70° C. | Multiple Suppliers |
| 96-well or 384-well cold block or ice | Multiple Suppliers |
| Nonstick, RNase-free microcentrifuge tubes (1.5 | Multiple Suppliers |
| mL and 2.0 mL) | |
| Sterile aerosol barrier (filtered) pipette tips | Multiple Suppliers |

### Use of Control Materials

A no-template control (NTC) should be run on every plate to detect reagent and/or environmental contamination. An NTC should consist of RNase/DNase-free water in place of a wastewater-extracted RNA specimen.

A GT-Variant Positive Control should be run to detect any variant-specific reagent failures and to aid in gridding the samples during analysis (see steps 14-16)

A GT-Parental Positive Control should be run to detect any Wuhan-specific reagent failures and to aid in gridding the samples during analysis (see steps 14-16).

A GT-Mixture—Control should be run to detect any multiplexing failures and to aid in gridding the samples during analysis (see steps 14-16).

### Protocol

1. Ensure extracted RNA sample(s) are stored on ice prior to analysis.

2. Bring the kit components to room temperature. Make sure to thaw the control materials (RNA) on ice.

3. Mix each component by brief vortexing to ensure homogeneity, then pulse centrifuge to collect contents at the bottom of the tube.

4. ddRT-PCR master mix preparation:
a. Prepare a master mix according to the number of samples and controls to be tested (Table 4)
b. Vortex the master mix briefly and pulse centrifuge to collect the contents to the bottom of the tube.

TABLE 4
ddPCR Master Mix Component Volumes

| | | Volume for 1 | Volume for 96 |
|---|---|---|---|
| | Volume for | sample (+4 | samples (+5 |
| | 1 sample | safety rxns) | safety rxns) |
| Component | (μL) | (μL) | (μL) |
| One-Step RT-ddPCR | 5.5 | 286 | 555.5 |
| Supermix | | | |
| Reverse Transcriptase | 2.2 | 114.4 | 222.2 |
| 300 mM DTT | 1.1 | 57.2 | 111.1 |
| GT-4-plex Primer-Probe | 1 | 52 | 101 |
| Solution | | | |
| RNA sample | 9* | n/a | n/a |
| Rnase/Dnase free water | 3.2** | 166.4 | 323.2 |

*Alternative RNA sample volumes can be run
**Adjust water for alternative RNA sample volumes

5. Add 13 uL of the master mix into the appropriate wells of a 96 well plate or PCR strip tube. Add 9 uL* of the extracted RNA sample per your planned plate layout.

*Alternative RNA sample volumes can be run, make sure to adjust compensating water volume.

6. Add 9 uL of the NTC, GT-Variant Positive Control, GTParental Positive Control, and GT-Mixture-Control to four separate wells reserved for controls in your planned plate layout.

We recommend keeping the plate or PCR tubes on ice or a plate cooler while loading.

7. Generate and transfer droplets per the manufacturer's recommendation to a 96-well ddPCR plate.

8. Heat seal the plate per the manufacturer's recommendation.

9. Place the sealed droplet-containing plate into the thermal cycler for reverse transcription and PCR amplification and run the following GT-RTddPCR Variant Test Thermal Cycling Protocol (Table 5).

TABLE 5
Recommended thermal cycling parameters

| | Temperature | | | Number of |
|---|---|---|---|---|
| Cycling Step | ° C. | Time | | Cycles |
| Reverse Transcription | 50 | 60 | min | 1 |
| Enzyme activation | 95 | 10 | min | 1 |
| Denaturation | 94 | 30 | sec | 45 |
| Annealing/Extension | 60 | 60 | sec | |
| Enzyme deactivation | 98 | 10 | min | 1 |
| Droplet Stabilization | 4 | 30 | min | 1 |
| Hold (optional) | 4 | 24 | hrs | 1 |

10. Upon thermal cycling completion, transfer the plate to the Droplet Reader.

11. Open QuantaSoft™ software and double click on any well to open Well Editor. Select the wells used in the run and choose the following:
a. Experiment: ABS
b. Supermix: One-step RT-ddPCR Kit for Probes
c. Target 1 Type: Ch1 Unknown
d. Target 2 Type: Ch2 Unknown

12. Add well-names and sample types if desired (optional).

13. Click Run, save template, and select FAM/HEX to begin reading.

### Analysis

14. When viewing data, please reference the example data below and use the three GT-Molecular provided controls for proper thresholding.
a. For the N501Y locus which is measured in Channel 1
   i. the high droplet population consists of droplets positive for the N501Y mutation
   ii. the middle droplet population consists of droplets positive for the Parental or parental sequence at the N501Y loci
   iii. the low droplet population consists of droplets with no target
b. For the Del69-70 locus which is measured in Channel 2 (HEX)
   i. the high droplet population consists of droplets positive for the Del69-70 mutation
   ii. the middle droplet population consists of droplets positive for the parental sequence at the del69-70 loci
   iii. the low droplet population consists of droplets with no target

15. In both channels, apply thresholds between the negative droplet populations and the middle Parental populations and export to .csv. This CSV contains data for all positive droplets.

16. In both channels, apply thresholds between the middle parental populations and the upper B.1.1.7 populations and export to .csv. This CSV contains data for the B.1.1.7 mutant population droplets for both targets.

17. Lastly, subtract the data exported in the second thresholding (B.1.1.7 mutant populations, step 16) from the first data export (all positive droplets, step 15) to isolate data for the Wuhan/Parental population.

An exemplary Plate Layout containing samples and various controls is provided in FIG. 20.

Proper aseptic technique should always be used when working with RNA. Always wear powder-free latex, vinyl, or nitrile gloves while handling reagents, tubes and RNA samples to prevent RNase contamination from the surface of the skin or from dust in the environment. Change gloves frequently and keep tubes closed.

During the procedure, work quickly and keep everything on cold blocks when possible to avoid degradation of RNA by endogenous or residual RNases. Clean working surfaces, pipettes, etc. with 20% bleach or other solution that can destroy nucleic acids and RNases.

The following Examples 5-6 represent illustrative output test results that can be communicated to an individual or entity who requested testing of a sample using a method or kit described herein. For example, relative amounts of detected variants can be provided.

### Example 5

### Hyper Transmissible SARS-CoV-2 Variant in Sample: Not Detected

Hyper Transmissible Variant Quantification: The alpha Variant of the B.1.1.7 lineage accounts for an alarming increase in cases in parts of England. Viruses in this lineage have an unusually large number of mutations, particularly in the Spike protein, which is the part of the virus that binds human cells and initiates infection. Our test does not test for every mutation present in the alpha variant, instead we target 2 key mutations that have been previously described to have biological effects that drive the hypertransmissibility of that alpha variant. The presence of both of these mutations is a strong indicator that the alpha variant or possibly another, related and yet to be defined hyper—transmissible variant is circulating within a community.

Mutation: Spike Protein del69-70: The deletion of amino acid residues 69 and 70 in the spike protein has been shown to cause a confirmation change to the spike protein and enhance viral infectivity and virus fitness.

Percentage of detected viruses with variant mutation: 0.000%

Parental Viral Copies per Liter wastewater at del69-70 location: 436,105 copies/L

Mutated Viral Copies per Liter wastewater at del69-70 location: 0 copies/L

Mutation: Spike Protein N501Y: Viruses containing the N501Y mutation demonstrate higher affinity for receptors found on human cells (2). This mutation, also found in the beta variant, has profound effects on infectivity and is potentially involved in immune system evasion (1).

Percentage of detected viruses with variant mutation: 0.000%

Parental (“wt”) Viral Copies per L wastewater at N501Y location: 432,650 copies/L Mutated Viral Copies per L wastewater at N501Y location: 0 copies/L

TABLE 6
Quality Control Data

| | Pass | | Pass or |
|---|---|---|---|
| Metric | Criteria | Measured | Fail |
| F+ Prophage Concentration | Detection | 2.62E+07 | Pass |
| (copies/L) | | | |
| Internal Process Control | >5% | 12% | Pass |
| (% Viral Recovery) | | | |
| ddPCR Positive Control | >20 | 2780 | Pass |
| (Copies/Rxn) | | | |
| ddPCR Negative Control | <1 | 0 | Pass |
| (Positive Droplets in NTC) | | | |

In this example, no viral sequences were detected with the key mutations that define the B.1.1.7 hyper transmissible lineage of viruses that includes the hyper-transmissible UK variant. This suggests there is a very low probability that this virus is circulating in your community at this time.

### Example 6

### Hyper Transmissible SARS-CoV-2 Variant in Sample: Detected

Hyper Transmissible Variant Quantification: The alpha Variant of the B.1.1.7 lineage accounts for an alarming increase in cases in parts of England. Viruses in this lineage have an unusually large number of mutations, particularly in the Spike protein, which is the part of the virus that binds human cells and initiates infection. 2 key mutations are targeted that have been previously described to have biological effects that drive the hypertransmissibility of that alpha variant. The presence of both of these mutations is a strong indicator that the alpha variant or possibly another, related and yet to be defined hyper-transmissible variant is circulating within a community.

Mutation: Spike Protein del69-70: The deletion of amino acid residues 69 and 70 in the spike protein has been shown to cause a confirmation change to the spike protein and enhance viral infectivity and virus fitness.

Percentage of detected viruses with variant mutation: 4.366%

Parental Viral Copies per Liter wastewater at del69-70 location: 743,749 copies/L

Mutated Viral Copies per Liter wastewater at del69-70 location: 32,475 copies/L

Mutation: Spike Protein N501Y: Viruses containing the N501Y mutation demonstrate higher affinity for receptors found on human cells (2). This mutation, also found in the beta variant, has profound effects on infectivity and is potentially involved in immune system evasion (1).

Percentage of detected viruses with variant mutation: 4.332%

Parental (“wt”) Viral Copies per L wastewater at N501Y location: 728,975 copies/L

Mutated Viral Copies per L wastewater at N501Y location: 31,580 copies/L

TABLE 7
Quality Control Data

| | Pass | | Pass or |
|---|---|---|---|
| Metric | Criteria | Measured | Fail |
| F+ Prophage Concentration | Detection | 2.62E+07 | Pass |
| (copies/L) | | | |
| Internal Process Control | >5% | 11% | Pass |
| (% Viral Recovery) | | | |
| ddPCR Positive Control | >20 | 2830 | Pass |
| (Copies/Rxn) | | | |
| ddPCR Negative Control | <1 | 0 | Pass |
| (Positive Droplets in NTC) | | | |

Both mutations of interest were detected, although at low levels, in this sample. This suggests there are strains in the B.1.1.7 lineage or other strains containing mutations that have been reported to confer increased transmissibility within your community.

### Example 7

### Exemplary Primer and Probes

TABLE 8
Primers and probes useful in the presently disclosed methods and kits

| Component | |
|---|---|
| GT 4-plex Primer-Probe | |
| Solution | |
| Subcomponent | |
| Item name | Item Sequence |
| N501Y Forward | CCGGTAGCACACCTTGTAAT (SEQ ID NO: 1) |
| N501Y Reverse | AGTTGCTGGTGCATGTAGAA (SEQ ID NO: 2) |
| del69-70 forward | CGTGGTGTTTATTACCCTGAC (SEQ ID NO: 5) |
| del69-70 reverse | ATGGTAGGACAGGGTTATCAA (SEQ ID NO: 6) |
| DEL69-70 PROBE | /5HEX/TACTTGGTT/ZEN/CCATGCTATCTCTGGGACC/ |
| | 3IABkFQ/ |
| N501Y_Probe_lna_ | /56-FAM/TGGTTTCCAACCCACT + TATGGTGT/3IABkFQ/ |
| version1 | (SEQ ID NO: 3) |
| N501Y_Probe_version2 | /56-FAM/TGGTTTCCAACCCACTTATGGTGT/3IABkFQ/ |
| | (SEQ ID NO: 28) |

Wastewater is received, filtered, and virus is concentrated using ultrafiltiration. RNA is then extracted from concentrated virus.

Concentrated RNA is then analyzed in replicate of 4 using the GT Molecular 4-plex RT-ddPCR kit for N501Y (Fam channel) and del69-70 (HEX channel)

Data is thresholded to obtain concentrations of wildtype and mutant in each channel and concentrations of each virus sequence is back calculated to starting wastewater concentration (unit=copies/L of wastewater) and ratios of wildtype to mutant sequence are calculated. All results are summarized in the example reports (one negative and positive) attached.

### Kit Composition:

This kit requires only 1 well to be run for each sample.

Single well analysis returns a parental (“wt”) measurement and a B.1.1.7 variant measurement (copies/uL) for 2 key B.1.1.7 mutations (del69-70 and N501Y)

### Kit Components:

GT-4-plex primer-probe solution*

TABLE 9
Kit Composition

| | Concentration |
|---|---|
| N501Y positive primer | 500 nM |
| N501Y reverse primer | 500 nM |
| N501Y Probe_LNA (FAM)* | 125 nM |
| Del69-70 forward primer | 500 nM |
| Del69-70 reverse primer | 500 nM |
| Del69-70 Probe (HEX) | 125 nM |

*although some configurations lack the LNA on the T at position 17(denoted as a + signed in the probe sequences above)

GT-Variant Positive Control**

Consists of synthetic B.1.1.7 viral RNA formulated to approximately 60 copies/uL

GT-Parental Positive Control**

Consists of synthetic Parental viral RNA formulated to approximately 60 copies/uL

GT-Variant-Parental Mix-Control**

Comprises synthetic Parental viral RNA formulated to approximately 60 copies/uL and synthetic B.1.1.7 viral RNA formulated to approximately 60 copies/uL.

### Example 8

### dPCR Delta-Omicron Varian Mutational Signature Assay for the Bio-Rad QX200 ddPCR® platform

The methods and assays provided herein may be used with available dPCR platforms, including the BioRad QX200 Droplet Digital™ system. See, e.g., www.gtmolecular.com/store-1/p/gt-rt-qper-sars-cov-2-variants-of-concern-mutational-signature-assay-kit-5nhg2-j28ng-nesm4-8548x-8arb9-rmf7h. The GT RT-dPCR Delta-Omicron Variant Mutational Signature Assay Kit (for QX200) is a molecular reagent kit containing all primers, probes, and controls for detection of S-gene mutations associated with the Delta (B.1.617.2) and Omicron (B.1.1.529) variant of SARS-CoV-2. While this test does not quantify every mutation present amongst the lineage, it targets key discriminating mutations associated with the Delta (L452R; T478K) and Omicron (N679K; Q954H) variants.

The kit includes one all-in-one primer probe solution and qualitative control sequences derived from GISAID accession numbers corresponding to the S-gene sequences of parental, Delta, and Omicron variants.

### Example 9

### RT-qPCR SARS-CoV-2, Delta-Omicron Varian Mutational Signature Assay for the Bio-Rad QX200 ddPCR® Platform

The methods and assays provided herein may be used with available RT-qPCR platforms, including with PBNJ. See, e.g., www.gtmolecular.com/store-1/p/gt-rt-qper-sars-cov-2-variants-of-concern-mutational-signature-assay-kit-5nhg2-j28ng-nesm4-8548x-8arb9-rmf7h, describing a RT-qPCR, SARS-CoV-2 Delta-Omicron Mutational Signature Assay Kit. The assay kit contains all primers, probes, and controls for detection of the signature mutations within the Spike protein-gene of the Delta (Indian, B.1.617.2) and Omicron (BA.1, BA.2, BA.3) variants. While this test does not target every mutation present in these virus variants, it targets variant-associated genomic sequences corresponding to Spike protein amino acids associated with the Delta (L452R and T478K) and Omicron (N679K and Q954H) variants. Kit includes validated GT-4-plex (L452R, T478K, N769K,Q954H) primer probe solution, plus validated, quantitative assay standards: GT-parental (parental) Standard, GT-Delta Standard and GT-Omicron Standard.

These examples further illustrate the methods and kits provided herein are compatible with both digital and quantitative real-time PCR. The RT-qPCR is preferable used with PBNJs to limit promiscuous probe hybridization to certain sequences, thereby further increasing discrimination between sequences.

Furthermore, probes with an LNA positioned at the SNP site dramatically increase specificity (see, e.g., FIG. 19). Probes also help discriminate not only viruses (such as SARS-CoV-2) SNP's, but also oncogene SNPs associated with cancer, such as KRAS G12C mutation), including with the use of promiscuity-blocking nucleotide juror oligonucleotide (PBNJ). See, for example, FIGS. 16-18. See also U.S. Pat. App. No. 63/271,522 filed Oct. 25, 2021 titled “OFF-TARGET BLOCKING SEQUENCES TO IMPROVE TARGET DISCRIMINATION BY POLYMERASE CHAIN REACTION” (Atty Ref. 339033: 97-21P US), which is specifically incorporated by reference herein. FIG. 16 illustrates a dose-response of a promiscuity-blocking nucleotide juror oligonucleotide (PBNJ) relative to probe in order to determine a concentration of PBNJ required to completely inhibit KRAS 12C nonspecific detection of the KRAS G12 sequence. Between 4×-8× PBNJ:probe ratio results in complete inhibition of nonspecific detection of KRAS G12 sequence. Accordingly, any of the methods and kits provided herein may be used with PBNJ to further reduce nonspecific amplification and/or detection.

FIG. 17 illustrates that PBNJ does not change the on-target detection, with concentration of mutant sequence that is independent of PBNJ concentration. FIG. 18 illustrates that 8× PBNJ completely removes nonspecific amplification.

### Example 10

### Cancer Mutations

Oligonucleotides are designed to detect cancer mutations for the presently disclosed methods and kits. Table 10 shows probes and primers for detecting an oncogene mutation, including in one or more of: BRAF600; KRAS, P53; EGFR.

TABLE 10
Probes and primers for detecting oncogene mutation

| Oligo Name | Sequence (5′→3′) | Modifications |
|---|---|---|
| BRAF_V600E_fwd | SEQ ID NO: 16 TGAAGACCTCACAGTAAA | |
| BRAF_V600E_probe | SEQ ID NO: 17 | FAM or |
| | TCTAGCTACAGAGAAATCTCGATGGAG | HEX, BHQ1 |
| BRAF_V600E_rev | SEQ ID NO: 18 CTGTCCAGTCATCAATTC | |
| KRAS_G12C_fwd1 | SEQ ID NO: 19 | |
| | TAAGGCCTGCTGAAAATGACTG | |
| KRAS_G12C_fwd2 | SEQ ID NO: 20 CACGTCTGCAGTCAACTG | |
| KRAS_G12C_probe | SEQ ID NO: 21 | FAM or |
| | CCTACGCCACAAGCTCCAACTA | HEX, BHQ1 |
| KRAS_G12C_rev1 | SEQ ID NO: 22 | |
| | GGTCCTGCACCAGTAATATGC | |
| KRAS_G12C_rev2 | SEQ ID NO: 23 | |
| | GCTGTATCGTCAAGGCACTC | |
| KRAS_G12C_fwd1 | SEQ ID NO: 29 | |
| | TAAGGCCTGCTGAAAATGACTG | |
| EGFR_L858R_fwd | SEQ ID NO: 24 CAAAGGGCATGAACTAC | |
| EGFR_L858R_probe | SEQ ID NO: 25 | FAM or |
| | TTTGGGCGGGCCAAACTG | HEX, BHQ1 |
| EGFR_L858R_rev | SEQ ID NO: 26 CACTCTGGTGGGTATAG | |
| p53_R175H_fwd | SEQ ID NO: 27 CCCTCAACAAGATGTTT | |
| p53_R175H_probe | SEQ ID NO: 30 | FAM or |
| | AGGTTGTGAGGCACTGCCC | HEX, BHQ1 |
| p53_R175H_rev | SEQ ID NO: 31 CTTCCACTCGGATAAGA | |

### Example 11

### Single Probe Multiplex

The platform described herein is also applicable to detection of three targets with one promiscuous probe. The assay can discriminate parental, alpha variant (the Parental and alpha variant are grouped together because they contain the same sequences at 417 and 484 loci), beta Variant, and gamma variants.

FIGS. 14-15 illustrate an assay developed using the promiscuous probes and related digital PCR described herein, including for K417N/T and E484K. In this example, the assay detects three nucleotide difference corresponding to the 417 amino acid locus in SARS-CoV-2 variants, including former variants of concern. In a similar manner, the platform provided herein is useful for detecting future arising variants of concern by the selection of promiscuous probes and primers relevant for the variant of concern. Table 11 provides the probes and primers sequences used for the assays summarize in FIGS. 14 and 15. In particular, the assay allows for detection and quantification of 417T, K417, 417N, E484, or 484K from parental, alpha, beta, or gamma variants.

TABLE 11

| Oligo Name | Sequence (5′→3′) | Modifications |
|---|---|---|
| K417N/T probe | SEQ ID NO: 32 | FAM, BHQ1 |
| | CAGGGCAAACTGGAACGATTGCTG | |
| E484K probe | SEQ ID NO: 33 | HEX, BHQ1 |
| | AGCACACCTTGTAATGGTGTTAAAGGTTT | |
| 417_fwd2 | SEQ ID NO: 34 | |
| | TGTAATTAGAGGTGATGAAGTCAGAC | |
| 417_rev | SEQ ID NO: 13 CAAGCTATAACGCAGCCTG | |
| 484-501_fwd2 | SEQ ID NO: 35 | |
| | CAAACCTTTTGAGAGAGATATTTCAACTG | |
| 484-501_rev2 | SEQ ID NO:36 TTGCTGGTGCATGTAGAAG | |

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects, exemplary aspects and optional features, modification and variation of the concepts herein disclosed may be resorted to by those skilled in the art, and that such modifications and variations are considered to be within the scope of this invention as defined by the appended claims. The specific aspects provided herein are examples of useful aspects of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific aspects that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

Unless otherwise explained, all technical and scientific terms used herein have the same meaning as commonly understood by one of ordinary skill in the art to which a disclosed disclosure belongs. The singular terms “a,” “an,” and “the” include plural referents unless context clearly indicates otherwise. Similarly, the word “or” is intended to include “and” unless the context clearly indicates otherwise. “Comprising” means “including”; hence, “comprising A or B” means “including A” or “including B” or “including A and B.” All references cited herein are incorporated by reference.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

### REFERENCES

[1] Rambaut, Loman, Pybus, et al., Preliminary genomic characterisation of an emergent SARS-CoV-2 lineage in the UK defined by a novel set of spike mutations. https://www.cogsconsortium.uk
[2] Kemp. SA, Havery, W T, Datir, R P, et al., Recurrent emergence and transmission of a SARS-CoV-2 Spike Deletion H69/V70., medRxiv

Table of Sequences:

| SEQ ID | | | Optional | |
|---|---|---|---|---|
| NO: | Oligo Name | Sequence (5′ -> 3′) | Cone | Label |
| 1 | N501Y forward primer | CCGGTAGCACACCTTGTAAT | 500 nM | |
| 2 | N501Y reverse primer | AGTTGCTGGTGCATGTAGAA | 500 nM | |
| 3 | N501Y | TGGTTTCCAACCCACT+TATGGTGT | 125 nM | FAM |
| | Probe_LNA_version | the base containing the LNA is | | |
| | | depicted with a ‘+’ | | |
| 4 | N501Y_Probe_version2 | TGGTTTCCAACCCACT | 125 nM | |
| | without LNA | | | |
| 5 | Del69-70 forward | CGTGGTGTTTATTACCCTGAC | 500 nM | |
| | primer | | | |
| 6 | Del69-70 reverse | CACCATCATTAAATGGTAGGACAG | 500 nM | HEX |
| | primer | | | |
| 7 | Del69-70 Probe | TACTTGGTTCCATGCTATCTCTGGGACC | 125 nM | HEX |
| 8 | 484-501_for | CCT GTA TAG ATT GTT TAG GAA GTC | 500 nM | |
| | | TAA TCT C | | |
| 9 | 484-501_rev | AGT TGC TGG TGC ATG TAG AAG | 500 nM | |
| 10 | 484K_1 | AGCACACCTTGTAATGGTGTTAAAGGTTT | 125 nM | 5’ FAM |
| | | | | and |
| | | | | 3’BHQ1 |
| 11 | 484K_2 | AGCACACCTTGTAATGGTGTTAAAGG | 125 nM | 5’FAM |
| | | | | and |
| | | | | 3’BHQ1 |
| 12 | 417 forward | AGGTGATGAAGTCAGACAAATCG | 500 nM | |
| 13 | 417 reverse | CAA GCT ATA ACG CAG CCT G | 500 nM | |
| 14 | 417T | CAG GGC AAA CTG GAA CGA TFG CTG | 125 nM | 5’FAM |
| | | AT | | and |
| | | | | 3’BHQ1 |
| 15 | 417N | CAG GGC AAA CTG GAA ACA TFG CTG | 125 nM | 5’FAM |
| | | AT | | or |
| | | | | 5’HEX, |
| | | | | 3’BHQ1 |
| 16 | BRAF_V600E_fwd | TGAAGACCTCACAGTAAA | | |
| 17 | BRAF_V600E_probe | TCTAGCTACAGAGAAATCTCGATGGAG | | FAM or |
| | | | | HEX, |
| | | | | BHQ1 |
| 18 | BRAF_V600E_rev | CTGTCCAGTCATCAATTC | | |
| 19 | KRAS_G12C_fwd1 | TAAGGCCTGCTGAAAATGACTG | 500 nM | |
| 20 | KRAS_G12C_fwd2 | CACGTCTGCAGTCAACTG | 500 nM | |
| 21 | KRASG12C_probe | CCTACGCCACAAGCTCCAACTA | 250 nM | FAM or |
| | | | | HEX, |
| | | | | BHQ1 |
| 22 | KRAS_G12C_rev1 | GGTCCTGCACCAGTAATATOC | 500 nM | |
| 23 | KRAS_G12C_rev2 | GCTGTATCGTCAAGGCACTC | 500 nM | |
| 24 | EGFR_L858R_fwd | CAAAGGGCATGAACTAC | | |
| 25 | EGFR_L858R_probe | TTTGGGCGGGCCAAACTG | | FAM or |
| | | | | HEX, |
| | | | | BHQ1 |
| 26 | EGFR_L858R_rev | CACTCTGGTGGGTATAG | | |
| 27 | p53_R175H_fwd | CCCTCAACAAGATGTTT | | |
| 28 | N501Y_Probe_ | TGGTTTCCAACCCACTTATGGTGT | | FAM |
| | version2 | | | |
| 29 | KRAS_G12C_fwd1 | TAAGGCCTGCTGAAAATGACTG | 500 nM | |
| 30 | p53_R175H_probe | AGGTTGTGAGGCACTGCCC | | FAM or |
| | | | | HEX, |
| | | | | BHQ1 |
| 31 | p53_R175H_rev | CTTCCACTCGGATAAGA | | |
| 32 | K417N/T probe | CAGGGCAAACTGGAACGATTGCTG | | FAM, |
| | | | | BHQ1 |
| 33 | E484K probe | AGCACACCTTGTAATGGTGTTAAAGGTTT | | HEX, |
| | | | | BHQ1 |
| 34 | 417_fwd2 | TGTAATTAGAGGTGATGAAGTCAGAC | | |
| 35 | 484-501_fwd2 | CAAGCTATAACGCAGCCTG | | |
| 36 | 484-501_rev2 | TTGCTGGTGCATGTAGAAG | | |
| 37 | G12 PBNJ + LNA | 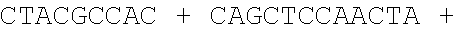 | | |
| | | indicates LNA | | |
| 38 | G12 PBNJ (no LNA) | CTACGCCACCAGCTCCAACTA | | |
| 39 | 452 fwd3 | CAG GCT GCG TTA TAG CTT GG | 500 | |
| 40 | 452R probe | TGA GAT TAG ACT TCC TAA ACA ATC | 250 | HEX, |
| | | TAT ACC GGT | | BHQ1 |
| 41 | 478K probe | TCAGGCCGGTAGCAAACCTTG | 125 | FAM, |
| | | | | BHQ1 |
| 42 | 679 fwd | TGT GAC ATA CCC ATT GGT GC | 500 | |
| 43 | 679 rev | GAT TGA CTA GCT ACA CTA CGT GC | 500 | |
| 44 | 679K probe | ATC AGA CTC AGA CTA AST CTC ATC GG | 250 | FAM, |
| | | | | BHQ1 |
| 45 | 954 fwd | CAA GAC TCA CTT TCT TCC ACA GC | 500 | |
| 46 | 954 rev | CCT CAA CTT TGT CAA GAC GTG A | 500 | |
| 47 | 954H_dd4-LNA probe | ATG TGG TCA ACC A+TA ATG CAC A | 250 | HEX, |
| | | | | BHQ1 |

1. A method for detecting the presence or absence of an at least one nucleotide difference in a target polynucleotide sequence, the method comprising the steps of:
a) providing a set of PCR primers comprising a forward amplification primer and a reverse amplification primer which, when hybridized to the respective primer annealing sites, flank the location of the target polynucleotide sequence, wherein the set of PCR primers are configured to generate in a PCR reaction a first amplicon comprising at least a portion of the target polynucleotide sequence and a second amplicon comprising at least a portion of the target polynucleotide with the at least one nucleotide difference;
b) providing a promiscuous probe that at a permissive temperature hybridizes at a first hybridization efficiency to the first amplicon and a second hybridization efficiency to the second amplicon, wherein the first hybridization efficiency is different than the second hybridization efficiency;
c) preparing a sample PCR reaction mixture comprising: a test sample having a sample polynucleotide, the PCR primers, the promiscuous probe, and PCR reagents;
d) performing at least one PCR reaction on the sample PCR reaction mixture at the permissive temperature to generate sample amplicons; and
e) measuring a fluorescence output generated by the promiscuous probe bound to the sample amplicons, wherein the difference between the first and second hybridization efficiencies results in a promiscuous probe fluorescence amplitude difference between promiscuous probe bound to target polynucleotide sequences with and without the at least one nucleotide difference;
thereby detecting the presence or absence of the at least one nucleotide difference in the target polynucleotide sequence.

2. The method of claim 1, wherein the measuring step further comprises quantifying the amount of the target polynucleotide sequence having the at least one nucleotide difference.

3. The method of claim 1, further comprising the steps of
preparing a positive control reaction mixture comprising the following constituents:
a positive control mixture comprising a first polynucleotide having the target polynucleotide sequence without the at least one nucleotide difference;
a positive control mixture comprising a second polynucleotide having the target polynucleotide sequence and the at least one nucleotide difference; and
a positive control mixture comprising the first polynucleotide and the second polynucleotide;
contacting each of the positive control reaction mixture constituents individually with the set of PCR primers and the promiscuous probe;
performing at least one PCR reaction on each of the contacted control reaction mixture constituents to generate a first and/or a second positive amplicon for each of the three constituent positive control reaction mixtures; and
validating the method by measuring a positive control fluorescence output generated by the promiscuous probe bound to the first and/or second positive amplicons, wherein a positive validation corresponds to positive clustering of fluorescence output.

4. The method of claim 3, further comprising the step of: defining a target threshold from the positive control mixture.

5. The method of claim 3, wherein the validating provides: (i) validation of each component of the method; and (ii) threshold definitions for each of the first polynucleotide and the second polynucleotide.

6. (canceled)

7. The method of claim 1, wherein the target polynucleotide sequence is from an organism selected from the group consisting of a virus, a bacteria, a fungus, a parasite, a plant cell, an animal cell, or a cancer cell.

8. The method of claim 1, wherein the target polynucleotide comprises RNA, and the method further comprises the step performing a reverse transcription reaction to produce a DNA target polynucleotide.

9. The method of claim 1, wherein the at least one nucleotide difference is from a mutation of one or more nucleotides in the target polynucleotide sequence, including an insertion mutation, a deletion mutation and/or a single nucleotide polymorphism (SNP).

10. The method of claim 1, wherein the target polynucleotide sequence has a length selected from a range of 60 bps to 1500 bps.

11. The method of claim 1, wherein the test sample comprises an environmental sample, soil, seed, plant material, wastewater sample, industrial water sample, natural water sample (including river, lake, stream, ocean, groundwater, well water, aquifer), a biological sample such as a gut/stool sample, a liquid or tumor biopsy from a cancer patient, a swab or saliva sample, an animal sample (veterinary/animal husbandry).

12. The method of claim 1, wherein the test sample is analyzed for short or single nucleotide polymorphisms (SNPs) whether associated with disease, drug resistance, multidrug resistance, herbicide resistance or not, insertions or deletions (indels) whether associated with a disease, the presence, absence, and/or abundance of viruses and viral variants, favorable or pathogenic bacterial, fungi, a non-invasive species, soil biome characterization (see if conducive/harmful to certain types of crops), and/or gut biome

13. The method of claim 1, wherein the promiscuous probe has a length that is between 20-35 bps without a locked nucleic acid or other melting temperature (Tₘ) increasing modification, or between 10-35 bps with a locked nucleic acid or other Tₘ increasing modification; the promiscuous probe optionally further comprising one or more of:
between 35%-80% GC content;
a Tₘ between 55° C.-62° C.;
a binding site to the at least one nucleotide difference that is positioned either: in a middle region of the promiscuous probe length, wherein the middle region is defined in a central 50% portion of the probe length, or at an alternative location at least partially outside the middle region so long as promiscuous binding at a permissive temperature is maintained; and/or
a locked nucleic acid at the at least one nucleotide difference.

14. The method of claim 1, wherein the promiscuous probe has a higher binding efficiency to the target polynucleotide sequence with the at least one nucleotide difference, or has a higher binding efficiency to the target polynucleotide sequence without the at least one nucleotide difference.

15. The method of claim 1, comprising a plurality of promiscuous probes for multiplex detection of a plurality of nucleotide differences in the target polynucleotide sequence.

16. The method of claim 1, wherein the promiscuous probe is a fluorescent or fluorescently-labelled probe, including a labelled probe comprising a locked nucleic acid.

17. The method of claim 1, wherein the promiscuous probe has greater than 98% binding region sequence complementary to a binding site of the target polynucleotide sequence for a high-hybridization efficiency condition, and less than 98% binding region sequence complementary to a binding site of the target polynucleotide sequence for a lower-hybridization efficiency condition.

18. The method of claim 1, wherein the promiscuous probe has a sequence configured to provide an at least 10% difference in amplitude of optical output for promiscuous probe bound to the first and second amplicons.

19. The method of claim 1, further comprising the step of determining the permissive temperature by:
contacting the positive control reaction mixture comprising an about 50:50 mixture of the first polynucleotide and the second polynucleotide with the primers and the promiscuous probe;
performing at least one PCR reaction on the control reaction mixture at a plurality of different temperatures spanning a low temperature that is below the permissive temperature and a high temperature that is above the permissive temperature; and
identifying a temperature or temperature range in which both the first and the second polynucleotides are amplified and optically detected with a magnitude shift of fluorescent output between the first and second polynucleotides due to lower efficiency off-target binding of the promiscuous probe compared to higher efficiency on-target binding of the promiscuous probe.

20. The method of claim 1, wherein the target polynucleotide sequence is from a SARS-CoV-2 virus, and the at least one nucleotide sequence difference corresponds to a variant of the SARS-CoV-2 virus.

21. (canceled)

22. (canceled)

23. The method of claim 20, wherein the variant comprises at least two mutations at different loci and the promiscuous probe comprises at least two promiscuous probes each having a distinct fluorescence emission maximum.

24. The method of claim 1, wherein the probes and primers comprise any one or more of:
SEQ ID NO:1, optionally at a concentration of approximately 500 nM;
SEQ ID NO:2, optionally at a concentration of approximately 500 nM;
SEQ ID NO:3 optionally at a concentration of approximately 125 nM;
SEQ ID NO:4 optionally at a concentration of approximately 125 nM;
SEQ ID NO:5 optionally at a concentration of approximately 500 nM;
SEQ ID NO:6 optionally at a concentration of approximately 500 nM;
SEQ ID NO:7 optionally at a concentration of approximately 125 nM;
SEQ ID NO:8, optionally at a concentration of approximately 500 nM;
SEQ ID NO:9, optionally at a concentration of approximately 500 nM;
SEQ ID NO:10 optionally at a concentration of approximately 125 nM;
SEQ ID NO:11 optionally at a concentration of approximately 125 nM;
SEQ ID NO:12 optionally at a concentration of approximately 500 nM;
SEQ ID NO:13 optionally at a concentration of approximately 500 nM;
SEQ ID NO:14 optionally at a concentration of approximately 125 nM;
SEQ ID NO:15 optionally at a concentration of approximately 125 nM;

25. (canceled)

26. The method of claim 1, having an at least 2-plex in one channel, with two channels in a single well, thereby providing a 4-plex per well.

27. The method of claim 1, further comprising:
discriminating single-nucleotide polymorphism (SNP), cancerous mutation, pathological mutation, a deletion mutation, an insertion mutation, drug resistance mutation, multi-drug resistance mutation, herbicide mutation, multi-herbicide resistance mutation, reassortment mutation, or a biomarker mutation.

28. (canceled)

29. The method of claim 1, wherein the target polynucleotide is obtained from a virus in wastewater, the method further comprising the steps of:
filtering the wastewater;
concentrating the virus;
extracting RNA; and
determining a relative concentration of a wildtype virus and a variant virus in the wastewater.

30. A method of making an assay to detect a first polynucleotide target sequence having a variant sequence differing from a second polynucleotide target sequence by at least one nucleotide, the method comprising the steps of:
a) identifying the first and second polynucleotide target sequences;
b) identifying an upstream flanking region and a downstream flanking region that are upstream and downstream from the polynucleotide target sequences;
c) designing a forward primer and a reverse primer that specifically bind to the upstream and downstream flanking regions of the first and second polynucleotide sequences, with a separation distance between the upstream and downstream primer binding regions that is between 50 bps and 1500 bps, wherein the primers are configured to generate a first and a second amplicon product corresponding to at least a portion of the first polynucleotide target sequence and at least a portion of the second polynucleotide sequence;
d) designing a promiscuous probe that will bind to the first and second amplicons with different hybridization efficiencies at a permissive temperature of between about 55° C. and 65° C.

31. The method of claim 30, wherein the design of primers is by selecting a flanking binding region of between 50 and 1500 nucleotides and the primer has at least 90% sequence complementarity to the flanking binding region.

32. The method of claim 30 or 31, wherein the design of the discriminatory probe comprises:
performing a thermal gradient dPCR on a target mixture comprising a mixture of the first polynucleotide target sequence and the second polynucleotide target sequence;
selecting a temperature of maximal separation of output fluorescent amplitudes between the first and second polynucleotide target sequence amplicons in the target mixture, thereby identifying for any promiscuous probe the permissive temperature.

33. The method of claim 30, further comprising the step of: performing the thermal gradient dPCR on: a first target that is 95%-100% a parent polynucleotide sequence; and a second target that is 95%-100% the variant polynucleotide sequence.

34. (canceled)

35. The method of claim 30 34, that is a 5-plex 417/484 assay for a SARS-CoV-2 virus and variants thereof, wherein the positive control mixture comprises approximately equal concentrations of parental, alpha, beta , or gamma variant polynucleotide sequences corresponding to the S gene.

36. A kit for distinguishing a first target polynucleotide sequence from a second target polynucleotide sequence by dPCR, wherein the first target polynucleotide sequence comprises at least one nucleotide difference not found in the second target polynucleotide sequence, the kit comprising:
a) for each location in the first target polynucleotide target sequence corresponding a nucleotide(s) difference: a set of PCR primers comprising a forward amplification primer and a reverse amplification primer which, when hybridized to the respective primer annealing sites, flank the location of the nucleotide(s) difference, wherein the set of PCR primers is capable of PCR amplification of corresponding regions of both the first polynucleotide target sequence and the second target polynucleotide sequence;
b) a labeled promiscuous probe designed at a permissive temperature to hybridize at a first hybridization efficiency to the site of the at least one nucleotide difference of the first polynucleotide and at a second hybridization efficiency to the corresponding site lacking the at least nucleotide difference of the second polynucleotide, wherein the first hybridization efficiency is different than the second hybridization efficiency;
c) a control mixture comprising a nucleic acid corresponding to the sequence of the first target polynucleotide;
d) a control mixture comprising a nucleic acid corresponding to the sequence of the second target polynucleotide; and
e) a control mixture comprising a nucleic acid mixture corresponding to the sequence of the first target polynucleotide and the sequence of the second target polynucleotide.

37. The kit of claim 36 for detecting a variant comprising primers and probes selected from the group consisting of:
a forward primer that is SEQ ID NO: 5-
(CGTGGTGTTTATTACCCTGAC);
a probe that is SEQ ID NO: 7
(TACTTGGTTCCATGCTATCTCTGGGACC);
a reverse primer that is SEQ ID NO: 6
(ATGGTAGGACAGGGTTATCAA);
a second forward primer that is SEQ ID NO: 1
(CCGGTAGCACACCTTGTAAT);
a second probe that is SEQ ID NO: 3
(TGGTTTCCAACCCACT + TATGGTGT);
and
a second reverse primer that is SEQ ID NO: 2
(AGTTGCTGGTGCATGTAGAA).

38. The kit of claim 37 for detecting an oncogene mutation, including in one or more of: BRAF600; TP53; EGFR, comprising one or more probes and primers selected from the group consisting of: SEQ ID NOs: 16-27.

39. The kit of claim 37 for detecting a herbicide resistance mutation in a plant gene.
